(19) United States
(12) Patent Application Publication
(10) Pub. No.: US 2026/0294429 A1
(43) Pub. Date: Oct. 1, 2026

(54) METHODS AND RELATED ASPECTS OF PERFORMING IMPACT FORCE-BASED MAGNETIC TISSUE PENETRATION

(71) Applicant: THE JOHNS HOPKINS UNIVERSITY, Baltimore, MD (US)

(72) Inventors: Önder ERIN, Baltimore, MD (US); Axel KRIEGER, Alexandria, VA (US); Xiaolong LIU, Baltimore, MD (US); Lamar MAIR, Bethesda, MD (US); Irving N. WEINBERG, Chevy Chase, MD (US); Trevor SCHWEHR, Baltimore, MD (US); Yancy DIAZ-MERCADO, College Park, MD (US)

(21) Appl. No.: 18/880,413

(22) PCT Filed: Jul. 3, 2023
(86) PCT No.: PCT/US2023/069567

§ 371 Date: Dec. 31, 2024

Related U.S. Application Data
(60) Provisional application No. 63/358,119, filed on Jul. 2, 2022.

(51) Int. Cl. A61B 17/06; A61B 17/00; A61B 34/00; A61B 34/32

(52) U.S. Cl. CPC A61B 17/06066; A61B 34/32; A61B 34/73; A61B 2017/00876

(57) ABSTRACT

Provided herein are methods of penetrating tissues using impact-force medical devices. Additional methods as well as related impact-force medical devices, kits, systems and computer readable media are also provided.

### CROSS-REFERENCE TO RELATED APPLICATIONS

This application is the national stage entry of International Patent Application No. PCT/US2023/069567, filed on Jul. 3, 2023, and published as WO 2024/011084 A8 on Jan. 11, 2024, which claims the benefit of U.S. Provisional Patent Application Ser. No. 63/358,119, filed on Jul. 2, 2022, which are hereby incorporated by reference in their entireties.

### BACKGROUND

State-of-the-art surgical robots (e.g., da Vinci Surgical System) enable minimally invasive procedures by providing surgeons with finer control and increased mobility via a few small incisions. However, these robotic manipulators have large mechanical footprints in the patient compared to the surgical end-effectors (e.g., needles and grippers), making the robotic manipulator the most invasive component involved in the procedure. Magnetically guided robotic systems have the potential to revolutionize surgical interventions by allowing tetherless access to magnetic tools operating inside the body. Using such a paradigm, magnetic fields could wirelessly guide magnetic agents (i.e., end-effectors) accurately and safely, obviating the mechanical manipulator and paving the way for untethered robotic surgical systems. Such a system may enable ultra-minimally invasive procedures, like the closing of a hole in the heart or repairing a hernia via insertion of only a needle and suture thread, as opposed to insertion of multiple trocars for manipulation of a needle and suture thread.

The size of a surgical tool or end-effector plays an important role in determining how minimally invasive a surgery could be. Magnetically guided end-effectors can be very small, as they are manipulated without contact and have no requirement to carry on-board power supplies or other electronics. However, magnetic pulling forces scale with L3 (L is the characteristic length of the magnetic object), while resistive forces such as drag forces scale with L2. As such, magnetic pulling forces are not dominant for small devices. Clinically relevant applications that include tissue penetration, biopsy, or suturing become either technically demanding or infeasible at such scales. Required forces can be achieved by bringing magnets very close to the end-effector, or increasing the power of electromagnets using greater coil currents. However, medical applications impose boundaries on how close external magnets can approach end-effectors for clinical usefulness, and large currents in electromagnets create difficulties due to resistive losses and heating. Thus, innovative methods of generating large end-effector force while maintaining tetherless operation have the potential to expand the magnetic surgery toolbox.

Magnetically actuated biopsy procedures have been demonstrated in vitro and ex vivo. Due to the high force requirements of these systems, unorthodox robot design methodologies have been developed. Vartholomeos et al. (The International Journal of Robotics Research 2013, 32 1536) have demonstrated a grounded gear train that is powered by the magnetic pulling forces generated inside of a magnetic resonance imaging (MRI) scanner. The grounded robotic system has dimensions 10×10×6 cm and is located outside of the patient. The biopsy needle can be pushed and pulled along a single desired direction in 3D space. In order to meet the high force requirements, this system uses a lever arm and a rotational mechanism connected to a gear train mechanism that results in a translational motion of the needle tip. As an alternative approach, untethered magnetic capsule-based biopsy operations can be performed only in the near vicinity of the electromagnetic actuators due to the high force requirements. However, methods for applying sufficient linear forces to clinical scale needles so as to enable tissue penetration or the basic functions of biopsy are still needed to advance the field. An alternative material engineering approach is demonstrated by Li et al. (Science Advances 2022, 8, 10 eabm5616) to provide high force outputs. The soft magnetic muscle material has been shown to respond radiofrequency (RF) heating and shrinks its size when the temperature is high. Such a shrink in size has been shown to provide high force delivery for drilling, suture and cutting applications. Permanent-magnet-based systems are developed to provide stronger forces. A set of magnets that consists of a small bore similar to Hallbach and Aubert arrays have shown the potential to exert stronger magnetic forces and torques. So far, these systems require the workspace to move (rotate and translate) inside the magnetic bore for a dexterous steering of magnetic robots. Moving the patient's bed continuously to accomplish complex navigation tasks could be a practical concern for medical operations, where the patient's bed should remain still. On the other hand, electromagnetic actuation systems do not have such a concern. However, many electromagnetic systems are unable to provide sufficient force due to the limitations in electrical power. Therefore, a complete penetration from outside of the sample with large penetration distances remains as a challenge for milli-sized miniature magnetic needles as shown in our previous study.

Recent studies have demonstrated the advantage of using magnetic impulse impacts to generate large instantaneous forces that suffice for various tissue penetration applications. The fundamental enabling principle in these studies is to magnetically slide a magnetic element inside of a larger container, allowing the magnetic element to gain sufficient velocity such that, upon striking an impact plate on one side of the container, the impact transfers the sliding magnet's momentum to the impact plate, inducing a momentary force on the entire device or at the needle tip. This technique has been used by Becker et al. (In 2015 IEEE International Conference on Robotics and Automation (ICRA). IEEE, 2015 1184-1189) to create an MRI-powered multi module Gauss gun. Once the robots get close enough to each other beyond the critical distance, the magnetic mechanism is triggered and a magnetic bead starts to accelerate and strikes a mobile needle tip. The momentum transfer onto the needle tip creates a projectile motion and the needle tip can go deep in the tissues. Quelin et al. (In 2021 IEEE International Conference on Mechatronics (ICM). IEEE, 2021 1-6) have used a similar impact method to create two-dimensional locomotion of a microrobot prototype that is impact driven. This system allows fine positioning of the chip with relatively smaller magnetic forces. Similarly, Leclerc et al. (IEEE Robotics and Automation Letters 2017, 3 403) demonstrated the use of such an impact force by developing a mono-directional magnetic hammer in which a 6 mm diameter sphere magnet is pulled and pushed inside a hollow tube, enabling penetration into ex vivo lamb brain.

However, these systems do not provide the degrees-of-freedom (DOF) motion capability that is required by most penetration applications for needle steering. For example, due to the strong and constant main magnetic field of an MRI device, the aforementioned Gauss gun and the MRI-powered hammer can penetrate only along a single direction. A full planar DOF is required for complete operation beyond straight line motion. Additionally, considering the centimeter-scale dimensions of these devices and their inability to orient to any desired penetration angle, such designs could be used only for biopsy along a single axis towards a single direction but could not be used for minimally invasive suturing applications that require 3-DOF, or interventions in the eye with approximately 25G size needle tip. Similarly, precise positioning of 25G needles is essential for clinical tasks such as hydrodissection and controlled intraocular access in cornea surgeries. During a cornea transplant, the surgeon must penetrate the eye, passing a needle up to 90 percent of way through the cornea thickness for best patient outcomes. Robots for autonomous cornea penetration are either too heavy to be eye-mountable, or are too slow for clinical use. Magnetically induced cornea penetration for intraocular access with a 25G needle may be one solution for robot assisted cornea surgeries as a light-weight and effective penetration solution.

Therefore, there is a need for additional methods, and related aspects, for magnetic robotic applications.

### SUMMARY

In one aspect, the present disclosure relates to an impact-force medical device. The device includes a body structure that comprises a channel disposed at least partially within the body structure, which channel communicates with first and second ends of the body structure, and a medical end-effector element directly or indirectly operably connected, or connectable, to at least the first end of the body structure. The device also includes an asymmetric magnetic element at least partially disposed within the channel, which asymmetric magnetic element is configured to selectively move within the channel at least when a magnetic field, pulse, and/or gradient is applied to the asymmetric magnetic element such that the asymmetric magnetic element reversibly applies an impulse force to the first or the second end of the body structure, which impulse force effects movement of the medical device.

In some embodiments, the channel comprises a cross-sectional shape selected from the group of: a circle, an oval, a regular n-sided polygon, and an irregular n-sided polygon. In some embodiments, the channel comprises a substantially cylindrical form. In some embodiments, the channel comprises dimensions that maximize a momentum of the asymmetric magnetic element at points of impact of the asymmetric magnetic element with the first and second ends of the body structure when the asymmetric magnetic element selectively moves within the channel.

In some embodiments, a ratio between a length of the asymmetric magnetic element and a length of the channel comprises between about 0.6 and about 0.7. In some embodiments, a length of the asymmetric magnetic element is about 12.1 mm and a length of the channel is about 18.1 mm. In some embodiments, the body structure comprises a substantially tubular form. In some embodiments, the medical device is configured to wirelessly operate as a component of a magnetic robotics system. In some embodiments, the medical end-effector element is configured to contact and/or penetrate in vitro, in vivo, and/or ex vivo tissue of a subject.

In some embodiments, the impulse force effects movement of the medical device in directions that are substantially parallel to a movement pathway of the asymmetric magnetic element within the channel at least proximal to the first or second ends of the body structure. In some embodiments, the asymmetric magnetic element comprises a permanent magnet. In some embodiments, the asymmetric magnetic element comprises a magnetizable material. In some embodiments, the asymmetric magnetic element comprises a cross-sectional shape selected from the group of: a circle, an oval, a regular n-sided polygon, and an irregular n-sided polygon. In some embodiments, the asymmetric magnetic element comprises a substantially cylindrical form. In some embodiments, the asymmetric magnetic element comprises a diameter of about 1.5 mm and a length of about 12.1 mm. In some embodiments, the asymmetric magnetic element comprises neodymium iron boron (NdFeB). In some embodiments, the asymmetric magnetic element is structured such that the medical device comprises two, three, four, five, or six degrees of freedom (DOF) when one or more magnetic fields, pulses, and/or gradients are applied to the asymmetric magnetic element.

In some embodiments, the impact-force medical device further includes an impact transmission structure operably connected to the body structure at least proximal to the first end of the body structure, wherein the impact transmission structure is operably connected, or connectable, to the medical end-effector element such that the medical end-effector element is at least indirectly operably connected, or connectable, to the first end of the body structure via the impact transmission structure and wherein the impact transmission structure is configured to transmit the impulse force to the medical end-effector element when the asymmetric magnetic element reversibly applies the impulse force to the first end of the body structure. In some embodiments, the first end of the body structure comprises a first opening that communicates with the channel, which first opening communicates with the impact transmission structure such that the asymmetric magnetic element contacts the impact transmission structure when the asymmetric magnetic element reversibly applies the impulse force to the first end of the body structure. In some embodiments, the impact transmission structure comprises an impact transmission plate.

In some embodiments, the medical end-effector element comprises a needle, a suture thread, a syringe, a blade, or a combination thereof. In some embodiments, the needle comprises a hypodermic needle that comprises a door structure that selectively moves between an open position and a closed position relative to a hollow opening of the hypodermic needle. In some embodiments, the body structure is substantially rigid. In some embodiments, the medical end-effector element comprises an asymmetric non-magnetic needle tip (e.g., a beveled needle tip or the like) having a needle tip axis, wherein the body structure is at least partially flexible, wherein the asymmetric magnetic element comprises a magnetic piston comprising a dipole that is oriented substantially parallel to the needle tip axis, and wherein the impact-force medical device further comprises a steering magnet structure connected at least proximal to the second end of the body structure, which steering magnet structure comprises a dipole that is oriented substantially perpendicular to the needle tip axis. In some embodiments, the body structure further comprises a second opening that communicates with the channel. In some embodiments, the impact-force medical device further includes a retaining structure operably connected, or connectable, to the body structure, which retaining structure is configured to prevent the asymmetric magnetic element from passing through the second opening of the body structure and to receive the impulse force from the asymmetric magnetic element to effect movement of the medical device when the asymmetric magnetic element reversibly applies the impulse force to the second end of the body structure. In some embodiments, the retaining structure comprises a cap that at least partially closes the second opening of the body structure.

In another aspect, the present disclosure provides a method of penetrating a tissue of a subject. The method includes positioning an impact-force medical device in a first position relative to the tissue of the subject using at least a first applied magnetic field, pulse, and/or gradient, wherein the impact-force medical device comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel. The method also includes contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

In some embodiments, the body structure is substantially rigid. In some embodiments, the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the method further comprises steering (e.g., turning or otherwise changing the direction of the needle tip while inside or penetrating the tissue) the medical end-effector element using at least the steering magnet structure. In some embodiments, the first applied magnetic field, pulse, and/or gradient and the second applied magnetic field, pulse, and/or gradient are different from one another. In some embodiments, the positioning step comprises moving the impact-force medical device using one or more translational motions and/or one or more rotational motions. In some embodiments, the positioning step and/or the contacting step comprises moving the asymmetric magnetic element into contact with the first end and/or the second end of the body structure one or more times. In some embodiments, the contacting step comprises moving the impact-force medical device entirely through the tissue of the subject. In some embodiments, the method further includes positioning the impact-force medical device in a second position relative to the tissue of the subject using at least a third applied magnetic field, pulse, and/or gradient. In some embodiments, the first applied magnetic field, pulse, and/or gradient and the second applied magnetic field, pulse, and/or gradient are different from one another.

In some embodiments, the medical end-effector element comprises a needle and a suture thread, and the method comprises suturing the tissue of the subject using the needle and the suturing thread. In some embodiments, the medical end-effector element comprises a needle and a syringe, and the method comprises delivering a therapeutic agent to the tissue of the subject using the needle and the syringe. In some embodiments, the medical end-effector element comprises a blade, and the method comprises making an incision in the tissue of the subject using the blade. In some embodiments, the medical end-effector element comprises a needle, and the method comprises obtaining a cell or tissue sample from the tissue of the subject using the needle. In some embodiments, a user of the impact-force medical device is at a location that is remote from the impact-force medical device.

In another aspect, the present disclosure provides a system that includes an impact-force medical device that comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel. The system also includes a magnetic coil subassembly configured to apply magnetic fields, pulses, and/or gradients at least to the asymmetric magnetic element. In addition, the system also includes a controller that comprises, or is capable of accessing, computer readable media comprising non-transitory computer-executable instructions which, when executed by at least one electronic processor, perform at least: positioning the impact-force medical device in a first position relative to a tissue of a subject using at least a first magnetic field, pulse, and/or gradient applied by the magnetic coil subassembly; and contacting the asymmetric magnetic element with at least the first end of the body structure one or more times with sufficient impact-force using at least a second magnetic field, pulse, and/or gradient applied by the magnetic coil subassembly such that at least the medical end-effector element at least partially penetrates the tissue of the subject.

In another aspect, the present disclosure provides a computer readable media comprising non-transitory computer executable instruction which, when executed by at least electronic processor, perform at least: positioning an impact-force medical device in a first position relative to a tissue of a subject using at least a first applied magnetic field, pulse, and/or gradient, wherein the impact-force medical device comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel; and contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

In some embodiments of the system or computer readable media, the body structure is substantially rigid. In some embodiments of the system or computer readable media, the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the non-transitory computer-executable instructions which, when executed by the electronic processor, further perform at least: steering the medical end-effector element using at least the steering magnet structure. In some embodiments of the system or computer readable media, the non-transitory computer-executable instructions which, when executed by the electronic processor, further perform at least: actuating the magnetic field to generate a torque on the impact-force medical device. In some embodiments of the system or computer readable media, the non-transitory computer-executable instructions which, when executed by the electronic processor, further perform at least: applying the magnetic field and/or pulse such that the impact-force medical device moves toward the magnetic coil subassembly. In some embodiments of the system or computer readable media, the non-transitory computer-executable instructions which, when executed by the electronic processor, further perform at least: accelerating the asymmetric magnetic element of the impact-force medical device toward the first or the second end of the body structure depending on a direction of the applied magnetic field using the magnetic coil subassembly. In some embodiments of the system or computer readable media, the non-transitory computer-executable instructions which, when executed by the electronic processor, further perform at least: adjusting a pulsing duty ratio of the applied magnetic field and a pulsing frequency of the applied magnetic field to maximize a momentum of the asymmetric magnetic element at points of impact of the asymmetric magnetic element with the first and second ends of the body structure when the asymmetric magnetic element selectively moves within the channel using the magnetic coil subassembly.

In some embodiments of the system or computer readable media, the non-transitory computer-executable instructions which, when executed by the electronic processor, further perform at least: applying a magnetic field pulsing sequence with a frequency of about 6 Hz and having a duty ratio of about 0.5 using the magnetic coil subassembly. In some embodiments of the system or computer readable media, the non-transitory computer-executable instructions which, when executed by the electronic processor, further perform at least: applying a magnetic field strength using the magnetic coil subassembly that accelerates the asymmetric magnetic element at points of impact of the asymmetric magnetic element with the first and second ends of the body structure having an impact force at least 20 times greater than when the medical end-effector element is only pulled using a same magnetic field strength. In some embodiments of the system or computer readable media, the non-transitory computer-executable instructions which, when executed by the electronic processor, further perform at least: generating a penetration force of the asymmetric magnetic element at points of impact of the asymmetric magnetic element with the first and second ends of the body structure of about 410 mN using the magnetic coil subassembly.

In some embodiments of the system or computer readable media, the channel comprises a cross-sectional shape selected from the group of: a circle, an oval, a regular n-sided polygon, and an irregular n-sided polygon. In some embodiments of the system or computer readable media, the channel comprises a substantially cylindrical form. In some embodiments of the system or computer readable media, the channel comprises dimensions that maximize a momentum of the asymmetric magnetic element at points of impact of the asymmetric magnetic element with the first and second ends of the body structure when the asymmetric magnetic element selectively moves within the channel. In some embodiments of the system or computer readable media, a ratio between a length of the asymmetric magnetic element and a length of the channel comprises between about 0.6 and about 0.7. In some embodiments of the system or computer readable media, a length of the asymmetric magnetic element is about 12.1 mm and a length of the channel is about 18.1 mm. In some embodiments of the system or computer readable media, the body structure comprises a substantially tubular form.

In some embodiments of the system or computer readable media, the medical end-effector element is configured to contact and/or penetrate in vitro, in vivo, and/or ex vivo tissue of a subject. In some embodiments of the system or computer readable media, the impulse force effects movement of the medical device in directions that are substantially parallel to a movement pathway of the asymmetric magnetic element within the channel at least proximal to the first or second ends of the body structure. In some embodiments of the system or computer readable media, the asymmetric magnetic element comprises a permanent magnet. In some embodiments of the system or computer readable media, the asymmetric magnetic element comprises a magnetizable material. In some embodiments of the system or computer readable media, the asymmetric magnetic element comprises a cross-sectional shape selected from the group of: a circle, an oval, a regular n-sided polygon, and an irregular n-sided polygon.

In some embodiments of the system or computer readable media, the asymmetric magnetic element comprises a substantially cylindrical form. In some embodiments of the system or computer readable media, the asymmetric magnetic element comprises a diameter of about 1.5 mm and a length of about 12.1 mm. In some embodiments of the system or computer readable media, the asymmetric magnetic element comprises neodymium iron boron (NdFeB). In some embodiments of the system or computer readable media, the asymmetric magnetic element is structured such that the medical device comprises two, three, four, five, or six degrees of freedom (DOF) when one or more magnetic fields, pulses, and/or gradients are applied to the asymmetric magnetic element.

In some embodiments, the system or computer readable media further include an impact transmission structure operably connected to the body structure at least proximal to the first end of the body structure, wherein the impact transmission structure is operably connected, or connectable, to the medical end-effector element such that the medical end-effector element is at least indirectly operably connected, or connectable, to the first end of the body structure via the impact transmission structure and wherein the impact transmission structure is configured to transmit the impulse force to the medical end-effector element when the asymmetric magnetic element reversibly applies the impulse force to the first end of the body structure. In some embodiments of the system or computer readable media, the first end of the body structure comprises a first opening that communicates with the channel, which first opening communicates with the impact transmission structure such that the asymmetric magnetic element contacts the impact transmission structure when the asymmetric magnetic element reversibly applies the impulse force to the first end of the body structure. In some embodiments of the system or computer readable media, the impact transmission structure comprises an impact transmission plate.

In some embodiments of the system or computer readable media, the medical end-effector element comprises a needle, a suture thread, a syringe, a blade, or a combination thereof. In some embodiments of the system or computer readable media, the needle comprises a hypodermic needle that comprises a door structure that selectively moves between an open position and a closed position relative to a hollow opening of the hypodermic needle. In some embodiments of the system or computer readable media, the body structure further comprises a second opening that communicates with the channel. In some embodiments, the system or computer readable media further include a retaining structure operably connected, or connectable, to the body structure, which retaining structure is configured to prevent the asymmetric magnetic element from passing through the second opening of the body structure and to receive the impulse force from the asymmetric magnetic element to effect movement of the medical device when the asymmetric magnetic element reversibly applies the impulse force to the second end of the body structure. In some embodiments of the system or computer readable media, the retaining structure comprises a cap that at least partially closes the second opening of the body structure.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIGS. 1A-1C: Magnetic suturing and tissue penetration with the benefit of impact forces. a.i) A typical procedure for hernia repair is to use a mesh and a suturing needle to close a defect. A magnetic needle controlled by external magnetic fields could accomplish this procedure remotely by revolutionizing the surgery with an ultra minimally invasive, magnetically controlled suture needle approach. a.ii) Tissue access, i.e. cornea access to eye, requires strong penetration forces from a needle. The proposed magnetic device could be able to provide transcorneal or transscleral access for various medical procedures. b) To overcome the force limitation for miniature magnetic robots, we designed and manufactured MPACT-Needle which utilizes a moving magnetic piston's momentum to realize momentarily high force outputs for tissue penetration. c) The running suture path to stitch a mesh into an agar gel and eye penetration are accomplished by the four electromagnetic coil system.

FIGS. 2A-2E: Impact-based needle design, impact mechanism with magnetic actuation sequence, and magnet length optimization. a) The impact-based magnetic needle consists of five main components: the needle tip, impact plate, tubular body, permanent magnet, and a cap. b) These components are assembled via cyanoacrylate-based adhesives or press-fit inside the tubular structure. The permanent magnet is slightly smaller than the diameter of the tubular structure and is sized so as to allow it to freely move back and forth within the tube. c) Increasing the length of the magnet increases the applied magnetic force but reduces the possible travelling distance within a limited tubular body. To maximize the impact force, the optimum size of the magnet is found to be 0.66 times of the overall tubular body length. d) The motile magnet is being pulled back and forth in the tubular body to create repetitive impact forces. The magnetic field created keeps the alignment of the magnet along the penetration direction. e) High speed recording snapshots of the collision moment and the overall magnet stroke.

FIGS. 3A-3F: Characterization experiments and scaling analysis. a) The forces are measured using a load cell under the workspace of the electromagnetic coil system. b) For a selected well-performing period duration, T=150 ms, the impact force measurements with respect to time are shown. The duty ratio, D, values ranging from 0.2 to 0.8 is swept with 0.1 increments. Having D in the range of 0.4 to 0.6 yields more than 400 mN momentarily forces in the needle. c) Selection of impractical D and T values results in degradation of the force performance. The optimal value for D and T is found to be 0.5 and 0.15 s for this study. d) Compared to the DC pulling force, the impact-based mechanism provides 22.7 times higher forces to allow penetration into tissue. e) Scaling of the needle dimensions are analyzed by manufacturing and experimentally measuring the impact force of each size of needle. The diameter and needle length are the two important factors in scaling. Larger the needle is, the more impact force we can acquire. f) Higher electrical power levels on the electromagnetic coils allow exerting stronger impact forces. MagnetoSuture™ System results in more than 400 mN forces for an optimized miniature needle dimensions at 14G.

FIGS. 4A-4M: Suturing on agarose gel with a gauze mesh. a) The experimental setup for demonstrating the suturing capacity on an agar gel: we prepared a clamp mechanism that holds the sample and allows mobility for the suturing needle. b) The agar gel is 0.6% and 2 mm in thickness. A gauze mesh is covered around the agar gel to represent the meshes being used in hernia repair. c-h) The needle is being steered by a handheld remote controller in an open-loop fashion. An overhead camera is being used to provide real-time monitoring of the workspace. The needle has demonstrated three penetrations in less than 3 minutes. j-m) The suture thread used for suturing the mesh and the agar gel is shown after the completion of the suturing task. The suture thread used for the experiments is 50 μm thick.

FIGS. 5A-5D: Eye penetration experiments. a) The holder structure used to stabilize the eye, mount the needle inside the coil system. b) The 25G needle being used with the impact force mechanism. c) The experimental snapshots of a penetration video. The MPACT-Needle is able to puncture the eye in less than 30.1 seconds. d) Images of the rabbit eye before penetration and after penetration by the MPACT-Needle. Due to the puncturing deep in the eye, a passage from the cornea to the deeper tissue layers is opened. This passage allows for a fluid exchange and delivering fluid drugs. We observe the leakage of the fluid from the inside of the eye towards the inside of the tube that holds the needle. A closer look reveals the location of penetration on the surface of the cornea.

FIGS. 6A-6E: a) Exemplary applications of Steerable MPACT-Needles involving cranial operations (left) and suturing (right) b) Steerable MPACT-Needle Design c) MagnetoSuture System used for testing d) Top down view of system e) Close up view of testing.

FIGS. 7A-7E: (A) Steerable MPACT-Needle composed of a sharp 18G MRI compatible needle tip, 3D printed end cap with inserted magnet, piston, and polyimide tube. Scale bar is 5 mm. (B) Needle is controlled using the MagnetoSuture system consisting of six individually addressable electromagnets with a central workspace. The top and bottom or “Z-Coils” are omitted for better visualization of the internal workspace. (C) Exploded view of needle assembly. In the figure, right-shaded area represents the north pole of the magnet and left-shaded area the south.

FIG. 8: A schematic of a rigid hammer needle.

FIG. 9: Setup and Progression of human in the loop target trial. Time is in minutes and seconds and scale bar is 1 cm.

FIG. 10: Results for bevel tip (top) and symmetric-tipped magnetic steering (bottom).

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Communicate: As used herein, “communicate” refers to the direct or indirect transfer or transmission, and/or capability of directly or indirectly transferring or transmitting, something at least from one area to another area.

In Some Embodiments: As used herein, the term “in some embodiments” refers to embodiments of all aspects of the disclosure, unless the context clearly indicates otherwise.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a respiratory disease, disorder, or condition, is going to receive a therapy for a respiratory disease, disorder, or condition, and/or has received at least one therapy for a respiratory disease, disorder, or condition.

System: As used herein, “system” in the context of analytical instrumentation refers a group of objects and/or devices that form a network for performing a desired objective.

### DETAILED DESCRIPTION

The field of magnetic robotics aims to obviate physical connections between the actuators and end-effectors. Such tetherless control enables new ultra-minimally invasive surgical manipulations in clinical settings. While wireless actuation offers advantages in medical applications, the challenge of providing sufficient force to magnetic needles for tissue penetration remains a barrier to practical application. Applying sufficient force for tissue penetration is needed for tasks such as biopsy, suturing, cutting, drug delivery, and accessing deep seated regions of complex structures in organs such as the eye. Accordingly, in some aspects, the present disclosure expands the force landscape for such magnetic surgical tools with an impact-force based suture needle capable of penetrating in vitro and ex vivo samples with 3-DOF planar motion. In some embodiments, for example, using custom-built 14G and 25G needles, we demonstrate generation of 410 mN penetration force, a 22.7-fold force increase with more than 20 times smaller volume compared to similar magnetically guided needles. With the MPACT-Needle, in vitro suturing of a gauze mesh onto an agar gel is demonstrated. In addition, in some embodiments, we have reduced the tip size to 25G, which is a typical needle size for interventions in the eye, to demonstrate ex vivo penetration in a rabbit eye, mimicking procedures such as corneal injections and transscleral drug delivery.

The invasiveness of state-of-the-art surgeries is affected by the tool, tool adapter, and robot arm size. Additionally, the necessary maneuverability of these tools in deep locations of the body also increases the invasiveness of the surgical operations. To overcome some of these limitations, as disclosed herein, magnetic fields can actuate the magnetic tool as the end-effector without any need for a physical connection to a robot arm. In some embodiments, the impact-force medical device or magnetic penetration devices disclosed herein are wirelessly powered rigid bodies that can push through tissues to penetrate towards deeper locations in the body. Unlike the devices disclosed herein, pre-existing magnetic penetration devices have been too large in size at least in part because providing the high magnetic forces needed for tissue penetration necessitated the use of relatively large magnetic components.

In some aspects, the present disclosure solves the issue related to the magnetic untethered robot size and the force trade-off, making these robots miniaturized into practical levels while maintaining penetration capability. Since force is proportional to the volume of magnetic materials, large forces need large magnetic fields and gradients for miniature magnetic devices. However, some medical interventions benefit from the controlled use of increasingly smaller devices. In some aspect, this disclosure solves the problem of applying larger forces to smaller devices by using an MPACT-Needle, as disclosed herein. In some embodiments, the mechanism presented herein offers a smart mechanism for producing 22.7-fold stronger forces for penetrating into tissues without any increase in the penetration needle size.

This feature brings advantages and solutions on at least four main aspects:
1. The need for further miniaturization: With this mechanism, the magnetic miniature tools can be miniaturized further, allowing for micro-scale magnetic tools in the body for medical applications. Using miniaturized magnetic tools reduces the tissue trauma or surgery side effects on the patients.
2. The need for electromagnetic systems with practical electrical power requirements: The electrical power needed for the electromagnetic coils can be brought to practical power levels. This removes a main technical concern along the way to the commercialization of such electromagnetic systems.
3. The need for the large workspace of magnetic fields: The working distance and region of interest of electromagnetic systems can be increased. This provides much larger volumes for clinical needs. This also leads to human-size magnetic manipulation systems for magnetic penetration becoming feasible.
4. The need for high penetration forces with magnetic tools: The increased benefit in the penetration force allows penetration into hard-to-penetrate tissues which were not feasible before with magnetic systems.

Due to the high force penetration capability of the devices and systems disclosed herein, one application of this technology is as a magnetic suture needle. This opens up a new surgical suture strategy, where only pin-prick access for the needle is sufficient for suturing. The needle can navigate inside the body and implement the suturing task on its own by the external magnetic fields. This enables ultra-minimally invasive magnetic suture applications. As another exemplary application, the devices of the present disclosure can also be used as untethered magnetic biopsy devices. For example, with sufficient forces, miniature biopsy tools can travel and acquire biopsy samples in the tissue. This strategy can be used especially for fine-needle-aspiration biopsy. In addition, devices that utilize various other biopsy techniques can also be adapted for use with the methods of the present disclosure. As another exemplary application, the devices disclosed herein can also be used as cleaning or removal tools for occluded or hard-to-remove plaque accumulations in the blood vessels. In some embodiments, with the guidance of catheters, a miniature magnetic tool is brought to a target site and the impact-force mechanism is initiated to effect plaque removal within blood vessels. Many other exemplary applications are also disclosed herein.

In some embodiments, the present disclosure provides an impact-force medical device that includes a body structure that comprises a channel disposed within the body structure. In some embodiments, the body structure has a substantially tubular form. The channel typically communicates with first and second ends of the body structure. The device also includes a medical end-effector element (e.g., a needle, scalpel, etc.) directly or indirectly operably connected to the first end of the body structure. In some embodiments, the medical end-effector element is configured to contact and/or penetrate in vitro, in vivo, and/or ex vivo tissue of a subject. In addition, the device also includes an asymmetric magnetic element (e.g., a permanent magnet) disposed within the channel. The asymmetric magnetic element is configured to selectively move within the channel when a magnetic field, pulse, and/or gradient is applied to the asymmetric magnetic element such that the asymmetric magnetic element reversibly applies an impulse force to the first or the second end of the body structure. This impulse force effects movement of the medical device to, for example, penetrate a targeted tissue. In some embodiments, the medical device is configured to wirelessly operate as a component of a magnetic robotics system.

In some embodiments, the channel has a cross-sectional shape selected from, for example, a circle, an oval, a regular n-sided polygon, or an irregular n-sided polygon. In some embodiments, the channel includes a substantially cylindrical form. In some embodiments, the channel comprises dimensions that maximize a momentum of the asymmetric magnetic element at points of impact of the asymmetric magnetic element with the first and second ends of the body structure when the asymmetric magnetic element selectively moves within the channel.

In some embodiments, a ratio between a length of the asymmetric magnetic element and a length of the channel comprises between about 0.6 and about 0.7 (e.g., 0.61, 0.62, 0.63, 0.64, 0.65, 0.66, 0.67, 0.68, 0.69, etc.). In some embodiments, a length of the asymmetric magnetic element is about 12.1 mm and a length of the channel is about 18.1 mm. In some embodiments, the impulse force effects movement of the medical device in directions that are substantially parallel to a movement pathway of the asymmetric magnetic element within the channel at least proximal to the first or second ends of the body structure. In some embodiments, the asymmetric magnetic element comprises a magnetizable material. In some embodiments, the asymmetric magnetic element comprises a cross-sectional shape selected from, for example, a circle, an oval, a regular n-sided polygon, or an irregular n-sided polygon. In some embodiments, the asymmetric magnetic element comprises a substantially cylindrical form (e.g., a rod shape). In some embodiments, the asymmetric magnetic element comprises a diameter of about 1.5 mm and a length of about 12.1 mm. In some embodiments, the asymmetric magnetic element comprises neodymium iron boron (NdFeB). In some embodiments, the asymmetric magnetic element is structured such that the medical device comprises two, three, four, five, or six degrees of freedom (DOF) when one or more magnetic fields, pulses, and/or gradients are applied to the asymmetric magnetic element.

In some embodiments, the impact-force medical device further includes an impact transmission structure operably connected to the body structure at least proximal to the first end of the body structure. The impact transmission structure is operably connected, or connectable, to the medical end-effector element such that the medical end-effector element is at least indirectly operably connected, or connectable, to the first end of the body structure via the impact transmission structure. The impact transmission structure is configured to transmit the impulse force to the medical end-effector element when the asymmetric magnetic element reversibly applies the impulse force to the first end of the body structure. In some embodiments, the first end of the body structure comprises a first opening that communicates with the channel. The first opening communicates with the impact transmission structure such that the asymmetric magnetic element contacts the impact transmission structure when the asymmetric magnetic element reversibly applies the impulse force to the first end of the body structure. In some embodiments, the impact transmission structure comprises an impact transmission plate.

In some embodiments, the medical end-effector element comprises a needle, a suture thread, a syringe, a blade, or a combination thereof. In some embodiments, the needle comprises a hypodermic needle that comprises a door structure that selectively moves between an open position and a closed position relative to a hollow opening of the hypodermic needle. In some embodiments, the body structure further comprises a second opening that communicates with the channel. In some embodiments, the impact-force medical device further includes a retaining structure operably connected, or connectable, to the body structure, which retaining structure is configured to prevent the asymmetric magnetic element from passing through the second opening of the body structure and to receive the impulse force from the asymmetric magnetic element to effect movement of the medical device when the asymmetric magnetic element reversibly applies the impulse force to the second end of the body structure. In some embodiments, the retaining structure comprises a cap that at least partially closes the second opening of the body structure.

In some embodiments, the medical end-effector element includes an asymmetric non-magnetic needle tip (e.g., a beveled needle tip or the like) having a needle tip axis, in which the body structure is at least partially flexible, in which the asymmetric magnetic element comprises a magnetic piston comprising a dipole that is oriented substantially parallel to the needle tip axis, and in which the impact-force medical device further comprises a steering magnet structure connected at least proximal to the second end of the body structure. The steering magnet structure typically includes a dipole that is oriented substantially perpendicular to the needle tip axis.

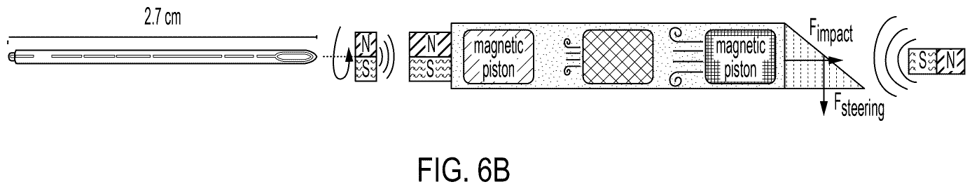

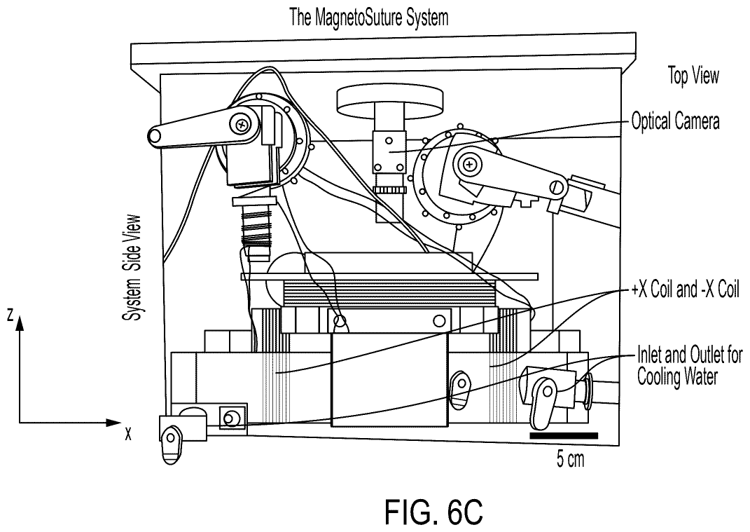

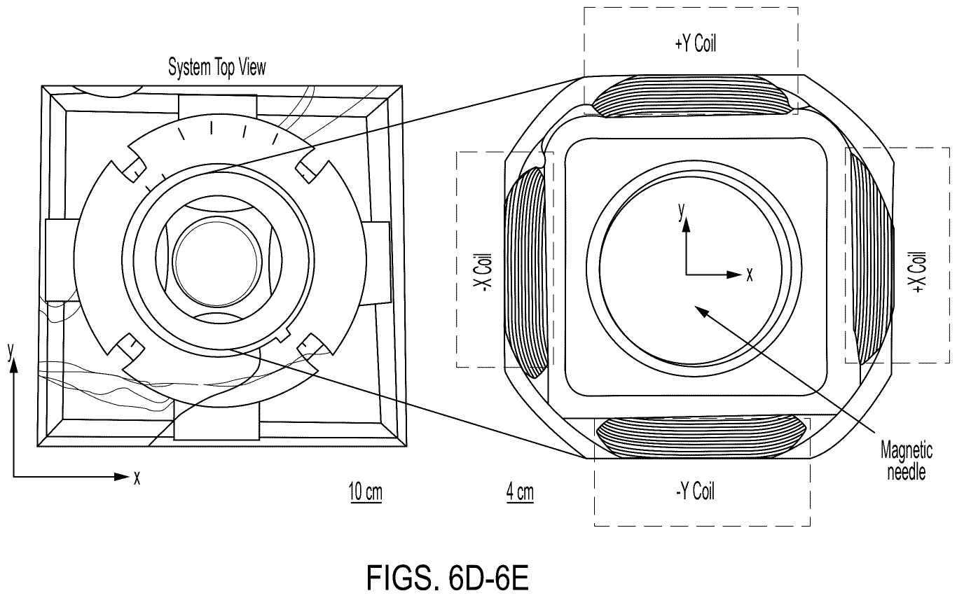

To further illustrate, in some embodiments, to allow for more complicated and realistic medical tasks, the MPACT-Needle is varied to be able to steer through, for example, stiff tissue to create a Steerable Magnetic Pulse Actuated Collisions for Tissue-penetrating Needle or Steerable MPACT-Needle. In some embodiments, this device includes an asymmetric, non-magnetic needle tip attached to a flexible tube with an enclosed magnetic piston and a rear steering magnet as shown, for example, in FIG. 6b. The magnetic piston placed inside the tube is oriented with the dipole pointing parallel to the needle axis, while the rear magnet is attached with the dipole perpendicular to the needle as shown by (N) representing north and (S) representing south. The Steerable MPACT-Needle is then controlled by, for example, the MagnetoSuture® system shown in FIG. 6 c, d, and e.

In some embodiments, to control the needle, the systems disclosed herein are able to propel the needle forward, but also change the heading of the needle tip and control the direction of tip deflection. To rotate the needle in the tissue, a rotating magnetic field is employed around the needle to create a torque on the rear magnet. This torque rotates the needle and allows for setting the heading of the asymmetric needle tip. The needle then moves along a curved path due to the perpendicular steering force shown in FIG. 6b. Continuous rotation of the needle while propelling forward causes the needle to move along a straight line. By controlling the rate of rotation, the systems cause the needle to follow various radii of curvature through tissue. This method is similar in certain respects to the method used by traditional steerable needles that consist of long flexible tubes or rods with asymmetric needle tip that are pushed at the distal end from outside of the body. The Steerable MPACT-Needle is able curve in a similar manner to traditional steerable needles due to the flexibility of the tube, but is also not tethered at the base unlike earlier devices, thus allowing for a smaller radius of curvature.

In some embodiments, a handheld controller is used to set the magnetic fields that cause the inner magnet to oscillate or the rear magnet to spin the needle. In this way, a surgeon can easily control the needle in real time. The controllability of the Steerable MPACT-Needle can also be expanded so that the surgeon can map out a path through safe areas of tissue and the needle will follow that path automatically. In some embodiments, this is implemented using a closed loop control or control that continuously checks the current position of the orientation of the needle within the workspace and makes adjustments in real time. Optionally, a camera is used for real time tracking of the needle in the MagnetoSuture® system as shown in FIG. 6. However, human tissue is opaque and more advanced methods can be used to measure the current state of the needle. There are a number of ways to check the status of the needle including, for example, ultrasounds, x-rays, or even using a grid of magnetic sensors that measure the magnetic field produced by the needle. In some embodiments, once a needle is located in the workspace, a control algorithm is used to steer the needle along the desired path. Real time sensing and control are also typically employed to make the Steerable MPACT-Needle suitable for clinical use.

The MPACT-Needle has the ability to drastically decrease the needed magnetic field for tissue penetration. By modifying the tip of the needle to be asymmetric and adding a rear steering magnet, the Steerable MPACT-Needle allows for complicated and realistic surgical procedures.

The present disclosure provides a method of penetrating a tissue of a subject. The method includes positioning an impact-force medical device in a first position relative to the tissue of the subject using at least a first applied magnetic field, pulse, and/or gradient. The impact-force medical device comprises a body structure that comprises a channel disposed at least partially within the body structure. The channel communicates with first and second ends of the body structure. The device also includes a medical end-effector element directly or indirectly operably connected to at least the first end of the body structure. In addition, the device also includes an asymmetric magnetic element movably disposed within the channel. The method also includes contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

In some embodiments, the first applied magnetic field, pulse, and/or gradient and the second applied magnetic field, pulse, and/or gradient are different from one another. In some embodiments, the positioning step comprises moving the impact-force medical device using one or more translational motions and/or one or more rotational motions. In some embodiments, the positioning step and/or the contacting step comprises moving the asymmetric magnetic element into contact with the first end and/or the second end of the body structure one or more times. In some embodiments, the contacting step comprises moving the impact-force medical device entirely through the tissue of the subject. In some embodiments, the method further includes positioning the impact-force medical device in a second position relative to the tissue of the subject using at least a third applied magnetic field, pulse, and/or gradient. In some embodiments, the first applied magnetic field, pulse, and/or gradient and the second applied magnetic field, pulse, and/or gradient are different from one another.

In some embodiments, the medical end-effector element comprises a needle and a suture thread, and the method comprises suturing the tissue of the subject using the needle and the suturing thread. In some embodiments, the medical end-effector element comprises a needle and a syringe, and the method comprises delivering a therapeutic agent to the tissue of the subject using the needle and the syringe. In some embodiments, the medical end-effector element comprises a blade, and the method comprises making an incision in the tissue of the subject using the blade. In some embodiments, the medical end-effector element comprises a needle, and the method comprises obtaining a cell or tissue sample from the tissue of the subject using the needle. In some embodiments, a user of the impact-force medical device is at a location that is remote from the impact-force medical device.

The present disclosure also provides a system that includes an impact-force medical device that comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel. The system also includes a magnetic coil subassembly configured to apply magnetic fields, pulses, and/or gradients at least to the asymmetric magnetic element. In addition, the system also includes a controller that comprises, or is capable of accessing, computer readable media comprising non-transitory computer-executable instructions which, when executed by at least one electronic processor, perform at least: positioning the impact-force medical device in a first position relative to a tissue of a subject using at least a first magnetic field, pulse, and/or gradient applied by the magnetic coil subassembly; and contacting the asymmetric magnetic element with at least the first end of the body structure one or more times with sufficient impact-force using at least a second magnetic field, pulse, and/or gradient applied by the magnetic coil subassembly such that at least the medical end-effector element at least partially penetrates the tissue of the subject.

The present disclosure provides a computer readable media comprising non-transitory computer executable instruction which, when executed by at least electronic processor, perform at least: positioning an impact-force medical device in a first position relative to a tissue of a subject using at least a first applied magnetic field, pulse, and/or gradient, wherein the impact-force medical device comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel; and contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

### EXAMPLES

### Example 1: Overcoming the Force Limitations of Magnetic Robotic Surgery: MPACT-Needle for Tetherless Interventions

### 1 Introduction

Here we present the design and evaluation of a Magnetic Pulse Actuated Collisions for Tissue-penetrating Needle (MPACT-Needle) to close the gap to achieving relevant levels of forces for magnetic needles and other magnetic miniature tools. This custom-built magnetic needle design brings the size-to-force trade-off into more favorable levels for the medical applications. The internal impact mechanism of the MPACT-Needle provides stronger penetration forces at small, clinically relevant size scales. For our MPACT-Needle, the impact mechanism yields 22.7-fold higher penetration forces at the moment of impact. In addition, we decrease the size of these impact-based devices by more than a factor of 20, and therefore, the volumetric footprint and weight of these devices are significantly reduced. Moreover, we demonstrate multi degrees-of-freedom (DOF) planar steering and manipulation (X- and Y-axis translation and in-plane rotation) instead of a single degree-of-freedom mechanisms. Therefore, the MPACT-Needle design results in an efficient use of electromagnetic systems, which have limits on power exertion, by transmitting the same magnetic energy into much stronger and efficient impact forces thanks to the sliding magnetic piston design.

We use our MPACT-Needle end-effector to demonstrate multiple surgically relevant tasks such as in vitro suturing and ex vivo eye cornea penetration for tissue hydrodissection and minimally invasive access. The requirements of a suturing task are typically prescribed by the sewing motion of the needle, requiring the needle to rotate and penetrate in various directions or at various angles—a capability we demonstrate here. Expanding on previous studies, we demonstrate 3-DOF steering of a needle that is oriented by torque-inducing magnetic fields and directed with magnetic gradient fields. This needle then operates in impact-momentum exertion mode via rapidly switching magnetic field gradients.

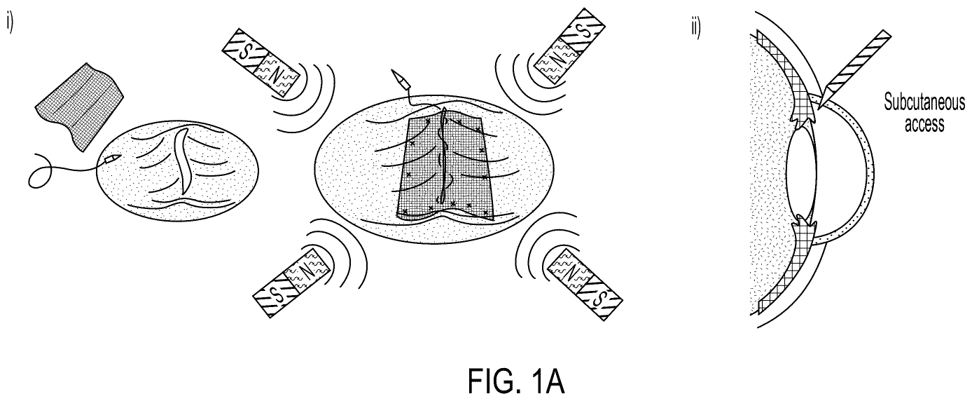

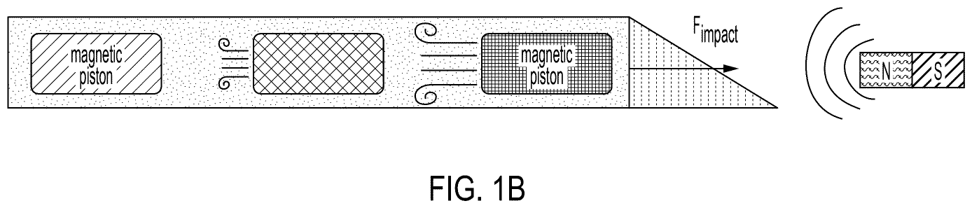

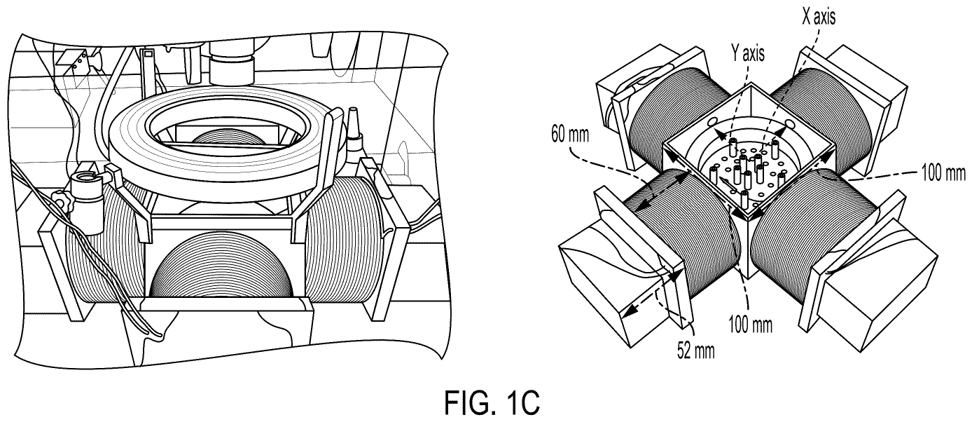

Conceptual depiction and the principal mechanism of the MPACT-Needle is demonstrated in FIG. 1. Magnetic suturing tasks and eye cornea penetration for tissue hydrodissection and minimally invasive access (demonstrated in FIG. 1a) are our primary clinically relevant applications for the MPACT-Needle. We utilize the electromagnetic system and the novel MPACT-Needle design to accomplish these tasks as depicted in FIG. 1bc. Additionally, we present a design optimization and experimental validation that maximizes the impact force generated at the needle tip and guides the selection of the magnetic piston length and the shaft length. We also present a scaling analysis for the needle dimensions (diameter and length) to show how the impact forces scale using mathematical computations and experimental measurements with various MPACT-Needle sizes. Along with the scaling analysis, an electrical power scaling analysis is also conducted to demonstrate the range of electrical power required for generating magnetic fields capable of various ex vivo and in vivo suturing experiments. Using our optimized design, we demonstrate a running suture implementation with in vitro agarose gel samples to move the MPACT-Needle concept closer to clinically relevant suturing tasks. Moreover, we present our ex vivo results on transcorneal penetration with a 25G needle tip to demonstrate the capacity in clinically relevant tasks.

### 2 Results

### 2.1 MPACT-Needle Design, Manufacturing, and Optimization

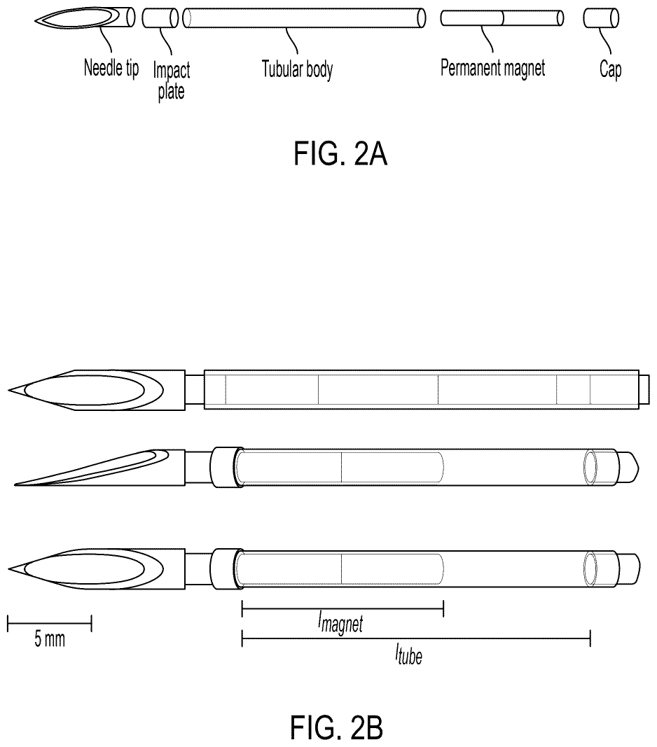

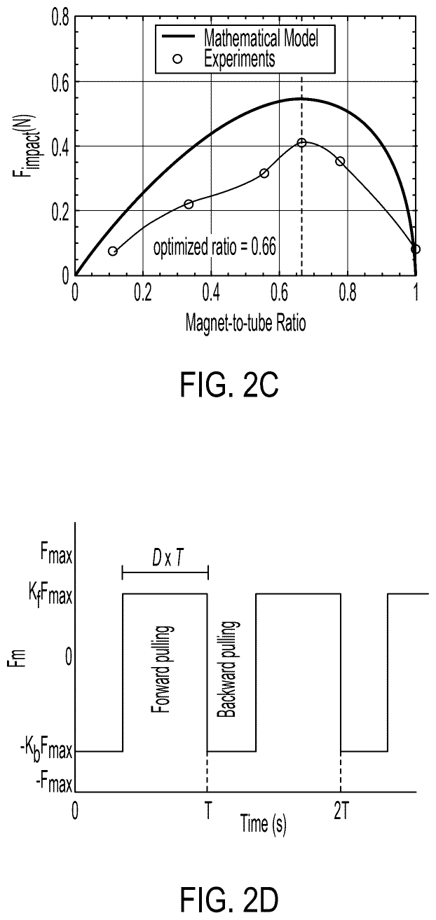

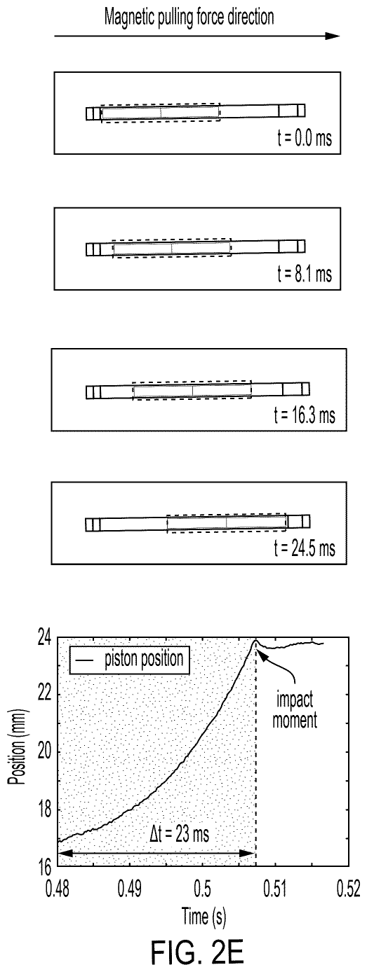

Needle Design and Manufacturing: The MPACT-Needle embodies the features of a suturing needle while incorporating a dynamically moving magnetic piston inside the needle's shaft. This MPACT-Needle design, optimization, and actuation sequence with impact mechanism is presented in FIG. 2. The MPACT-Needle, shown in FIG. 2a-b, is comprised of five components: 1) a needle tip, 2) a tubular body, 3) a permanent magnet, 4) an impact plate, and 5) a cap. To provide strong forces and the ability to suture while keeping the footprint small, we selected a 14G needle diameter as a compromise amongst needle manufacturability, medical relevance, and attainable force. Needing only the sharpened hypodermic needle tip, we cut 9 mm from the tip of a standard tribeveled standard hypodermic needle (14G puncture needle, TC INC). For suturing application, the needle tip size used is 14G; for eye penetration, we used a 25G hypodermic needle tip. The permanent magnet is a Nd—FeB magnet (Cyl0050, Supermagnetman) that has a cylindrical shape of 1.5 mm diameter and 12.1 mm length. These magnets are inserted into the MPACT-Needle's tubular body, which is made of Polytetrafluoroethylene (PTFE) plastic (75175A671, McMaster-Carr) that is 18.4 mm long with an outer diameter of 2.08 mm and inner diameter of 1.67 mm. The impact plate and cap were composed of a glass-mica (Macor®) cylindrical rod composite (8489K11, McMaster-Carr), both having identical dimensions of 3 mm length and 1.6 mm diameter. The magnet diameter is selected as slightly smaller than the tube's inner diameter to maximize the magnetic force (NdFeB volume) while still allowing ample space for the magnet to accelerate in the hollow shaft, as well as ample clearance for the magnet to slide smoothly along the interior of the needle shaft.

The impact plate is designed to efficiently deliver the impact force from the magnet to the needle tip, ideally with as little force loss as possible. Therefore, a Macor® ceramic was chosen for its stiffness, impact resistance, and machinability. Moreover, since glass-mica ceramic is a non-conductive material, the potential eddy-current generation and the resulting magnetic breaking phenomena are avoided. Both the impact plate and cap are assembled via a press fit to the plastic tubular body. The needle tip is attached to the impact plate using a cyanoacrylate based adhesive. A similar adhesive is used for attaching the suture thread at the tail of the needle.

Magnetic Piston Design Optimization: Maximizing the impact force while minimizing the length of the needle shaft is the main goal of the design optimization. Increasing the length of the magnet increases the magnetic pulling force, Fₘ, and the magnet mass, m. However, the travelling distance from tail to the tip of the needle decreases, allowing less distance for build-up of momentum. While a larger mass indicates a stronger impact momentum, shorter travelling distances result in smaller velocity magnitudes and weaker impact momentum. Therefore, there is an optimum ratio between the length of the magnetic piston and the tube length to maximize the impact force. Therefore, the optimization problem can be defined as

$${maximize}_{{l}_{magnet}} {f}_{impact} ( {l}_{magnet} ) \qquad ( 1 )$$

$$subject to {l}_{magnet} \leq {l}_{tube}$$

where f_{impact}(l_{magnet}) is the objective function, l_{magnet} is the length of the magnet, l_{tube} is the length of the tube, Defining k=l_{tube}−l_{magnet} as the travelling length of a magnet from the tail to the tip of the needle and considering Equation (6), (7) and (8), the impact momentum under a constant acceleration towards the tip can be defined as l_{magnet}\sqrt{2ak}. To maximize the impact force, this impact momentum should be maximized. Defining constants under a single constant parameter,

$$C = {ρ}_{m} π {r}_{m}^{2} \sqrt{2 a} ,$$

yields the objective momentum function as Cl_{magnet}\sqrt{(k)}. Replacing k=l_{tube}−l_{magnet} yields the optimization function as

$${maximize}_{{l}_{magnet}} f ( {l}_{magnet} ) = {Cl}_{magnet} \sqrt{( {l}_{tube} - {l}_{magnet} )} . \qquad ( 2 )$$

$$\frac{df ( {l}_{magnet} )}{{dl}_{magnet}} = 0$$

yields the optimum final relation as

$${I}_{magnet} = \frac{2 {I}_{tube}}{3} = 0.66 {I}_{tube} . \qquad ( 3 )$$

By setting l_{tube}=18.4 mm for the needle proposed in this study, the optimum l_{magnet} was found to be 12.1 mm. We have experimentally and mathematically swept the magnet-to-tube ratio as shown in FIG. 2c, and pinpointed the optimized ratio as 0.66 experimentally as well. For experimental testing, various MPACT-Needles with different l_{magnet} sizes are manufactured by sweeping the l_{magnet} parameter from 2 mm to 18 mm while keeping the l_{tube} constant at 18.4 mm. As shown in FIG. 2c, a non-optimized design could result in a significant reduction of the maximum impact force accomplishable by the needle, making the penetration infeasible. For the range of experimental measurements, we found out that a non-optimum magnetic piston length selection may result in a factor of 9.7 less impact force than the optimized version. The equivalent impact force is the summation of the pulling force and momentum transfer-based force. The magnetic pulling force and manufacturing errors (sub-millimeter gaps in the tube for the magnet to travel) result in nonzero impact force results for the unity magnet-to-tube ratio case.

### 2.2 Impact Mechanism and Time-Dependent Parameters

The cylindrical permanent magnet can freely move inside the tubular body back and forth. The magnetic field is sequentially generated to pull and push this magnetic piston inside the tube while preserving the magnetic alignment. Forward pushing results in collision of the magnetic piston with the ceramic impact plate. This collision generates a sudden impact force at the moment of the impact. Amount of this force is much stronger than the force that can be accomplished by pure magnetic pulling. Therefore, needle penetration requiring tasks with miniaturized tool sizes becomes possible for magnetic robotics. FIG. 2d defines this alternating pushing and pulling actuation sequence. The performance of the overall impact force depends on the period of the sequence, T, duty ratio of the forward motion, D, as well as forward and backward pulling force constants, K_{f} and K_{b}, respectively. To maximize the impact force, the strongest pulling force should be applied (i.e., K_{f}=1), and the NdFeB magnet should travel the longest possible distance. That is, ideally, the magnet starts at the tail of the shaft and travels the entire distance along the tube, striking the impact plate and remaining in contact with the impact plate until all magnet momentum is transferred. Hence, the selection of both D and T determines the impact force performance. Improper selection of parameters D and Twill result in incomplete or inefficient transfer of force from the magnet to the impact plate. This could happen either because the magnet does not transfer momentum completely, or because the magnet does not reach the impact plate due to premature stopping. Similarly, the backward force must also be tuned properly such that the magnet travels backward, recovering the travelling distance for subsequent, cyclic impact events without causing a considerable backward impact force. To visualize the impact moment, the needle tube is attached on top of a double-sided tape and a high speed camera (Photron, FASTCAM SA5, San Diego, CA, USA) is mounted on top of the workspace. FIG. 2e demonstrates the high-speed camera snapshots of the impact moment as well as the piston position tracking for this specific stroke. The magnet travels from one end to the other end in 23 ms, transfers the momentum, and shows a slight bounce back since the tube is rigidly attached to the double sided tape.

### 2.3 Impact Force Characterization and Scaling of the Robotic System

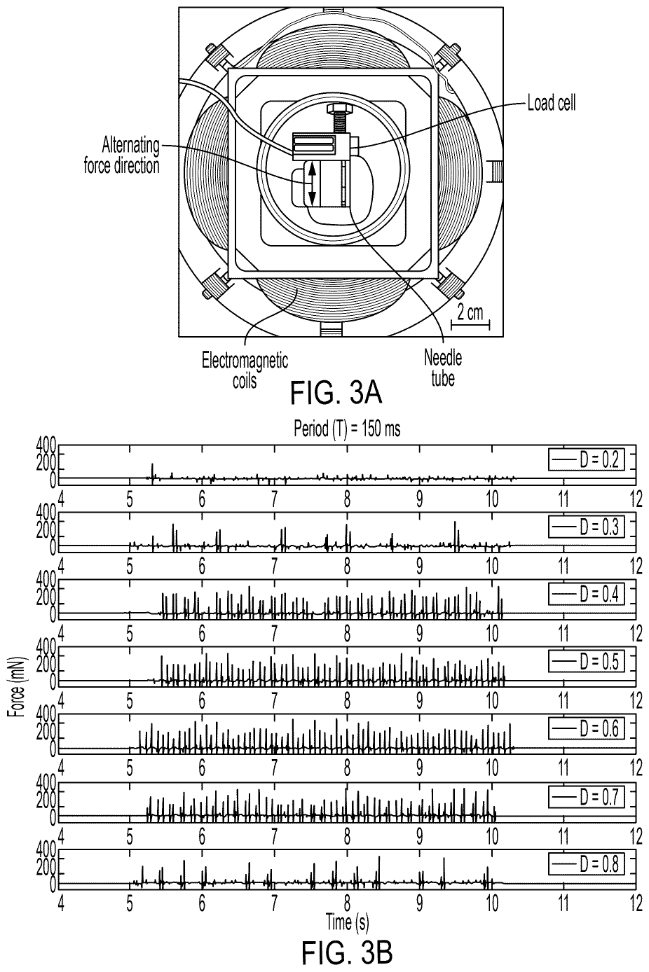

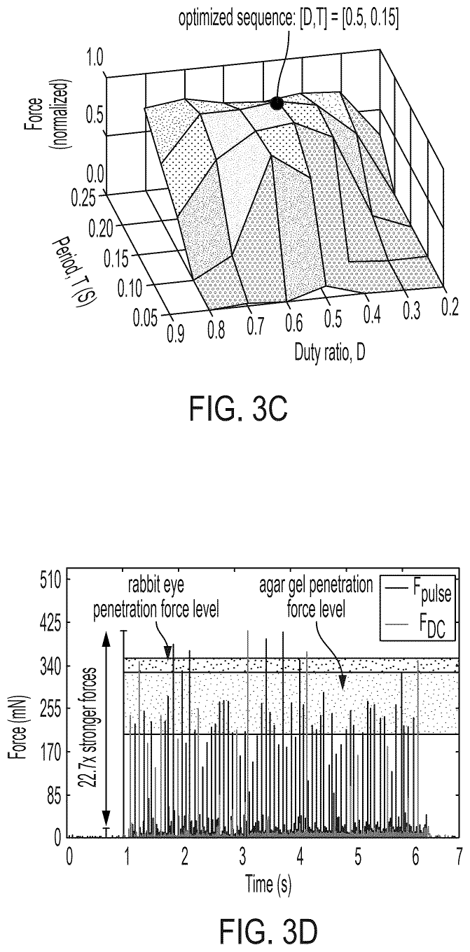

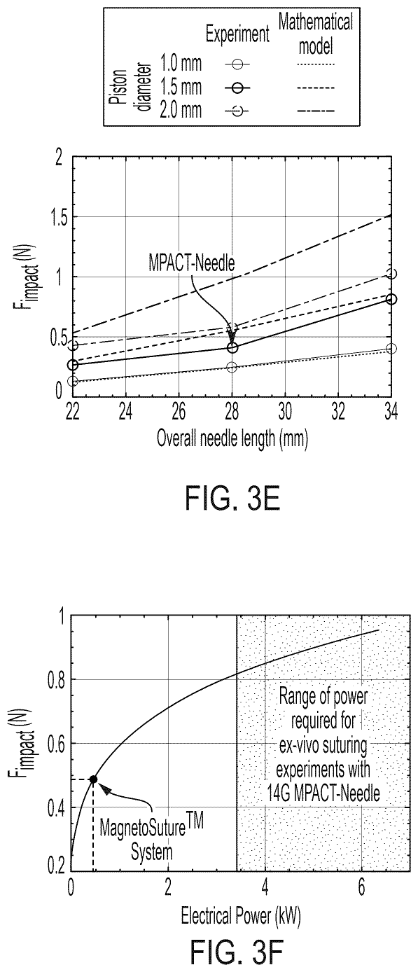

Characterization of Pulsing Sequence: We have experimentally measured the impact force by sweeping D and T to find the optimal actuation configuration to maximize the net force. We swept D from 0.2 to 0.8 with 0.1 increments. Similarly, we swept T from 0.05 s to 0.25 s in 0.05 s of increments. FIG. 3 presents the experimental measurement system, results of the experiments as well as theoretical scaling analysis for the needle and the system. For the experiments, we used a load cell (Transducer Techniques, GSO-1K) and located the tube at the center of the workspace as shown in FIG. 3a. We applied the pulsing sequence by having a two-dimensional sweep for D and T variables while recording the measured force in real time. Experimental results (FIG. 3b-c) indicate that, for the highest and the most repetitive impact mechanism, the ideal period of the sequence, T, was found to be 0.15 s, while the ideal duty ratio D was 0.5 when the needle was at the center of the workspace. It should be noted that T varies depending on the needle location inside the petri dish due to the nonhomogeneous magnetic gradient distribution. For the cases when the needle is far from the center, the user could manually tune K_{f} and K_{b} constants to ensure proper operation of the impact mechanism. Reducing the forces with K_{f} and K_{b} would result in larger T values depending on the deviation from the center. We used these experimentally acquired optimization values for T and D for force comparison between typical magnetic pulling forces and impact forces of MPACT-Needle. We measured both the pulsing impact force and DC gradient pulling force to determine force values, each measurement being performed three times. The acquired data was used to compare the impact-based penetration force with the typical magnetic pulling force without any impact for the same needle. To acquire the DC pulling force for our design, we applied and measured the DC pulling force when the magnetic piston is in contact with the impact plate already. The same setup and the same magnetic device were used for measuring the impact forces for the optimized actuation sequence. Both DC force measurements and impact force measurements with respect to time are provided in FIG. 3d to provide a better contrast with the force amplification accomplished with the impact-force mechanism. Since the DC pulling force should be constant under a constant pulling force, the DC pulling force is determined by taking the mean of the accumulated data over 6 seconds. On the other hand, since the impact force is a momentarily burst of force, we have taken the mean of the highest peaks of three strokes to demonstrate an achievable magnetic forces. This comparison has shown that, while the impact-force based mechanism can provide up to 410 mN of force for the optimal magnet-to-tube ratio (0.66), the continuous gradient pulling force mechanism can only provide 18 mN of force at the center of the workspace (FIG. 3d). According to the microscopy (UM1000, AMScope) image measurements, we found that the needle tip is 0.11 mm thick and 0.032 mm wide, resulting in a tip area of 0.0035 mm². This would result in 117 MPa pressure applied at the very needle tip at each impact applied.

Needle Size Scaling: In order to understand the trend and expected impact force values as a function of needle diameter and length, we have calculated the impact force on the needle for various dimensions of the tube geometry. While maintaining the optimized magnet-to-tube ratio constant (described in the Design Optimization subsection under Materials and Methods), we have scaled the needle length and the needle diameter. The magnetic piston length and its diameter are scaled along with the dimensions of the needle. The impact force, f_{impact}, is calculated with the assumption of perfect momentum transfer, which is a reasonable approach since we use Macor® impact plate rather than a soft plastic material. The impact force is assumed to be constant over the duration of the impact, Δt_{impact}, which is experimentally measured as 400 μs with a load sensor. Since the friction force on the magnetic piston is orders of magnitude smaller than the magnetic pulling force, we neglect the effect of friction force. With the constant acceleration of the piston before the impact with the magnetic pulling force, Fₘ, the resultant impact force, f_{impact}, can be calculated as

$${f}_{impact} = m \frac{\sqrt{2 \frac{{F}_{m}}{m} k}}{Δ {t}_{impact}} , \qquad ( 4 )$$

where k is the net distance the piston can travel: k=l_{tube}−l_{magnet}. Fₘ, m, and k depend on the needle size scaling and each of these terms scale as L³, L³, and L, respectively (further details are provided under Materials and Methods Section). Thus, according to Equation 4, the overall scaling law of the impact force is found to be proportional to L^{3.5}. This indicates that the impact force mechanism is preferable at larger scales and may require more magnetic power or adapting the electromagnetic setup size for smaller scales. The trend can also be seen in FIG. 3e.

To complement the theoretical findings with experiments, we have manufactured various sizes of MPACT-Needle bodies to experimentally explore the relationship between needle size, length, and impact force. While keeping the optimized magnet-to-tube length ratio constant, we have used 8, 12, and 16 mm as the magnetic piston lengths, and 1.0, 1.5, 2.0 mm as the diameters of the magnetic pistons, resulting in a total of 9 different MPACT-Needle geometries. The experimental results of the scaling analysis show a similar trend with the mathematical model of Equation 4 with some intensity mismatch between the experimental measurements and mathematical estimations (FIG. 3e). The error in experimental measurements derives from multiple sources, including variations in friction between the piston and the inner tube, slight sample positioning mismatches, magnetic field modeling inaccuracy, and custom-made needle manufacturing errors. Assumptions in the mathematical model such as perfect momentum transfer and constant impact force during the impact event are also potential contributors to the discrepancies between experimental measurements and the mathematical model.

Electrical Power Scaling: Our electromagnetic coil system can exert a finite force limited by the power train, motor controllers, and coil geometry. Based on the mathematical model shown in Equation 4, we estimated the impact force with respect to the power applied on the electromagnetic coils (FIG. 3f). The result shown in FIG. 3f indicates a nonlinear relation between the electrical power and the impact force. In our experimental measurements and trials for the penetration of a bacon strip, chicken tissue, and rabbit abdominal wall, the penetration force requirements suggest that for the current MagnetoSuture™ System, more than 7.5 times the available electrical power is required for a magnet at the center of the workspace (approximately at a distance of 5 cm from the electromagnetic coil surface to the center of the needle magnet). Even though the power and the impact force do not scale linearly, 3.5 kW or more electrical power would bring the penetration of the MPACT-Needle into the force range for ex vivo suturing applications.

2.4 In Vitro Suturing and Ex Vivo Eye Penetration with MPACT-Needles

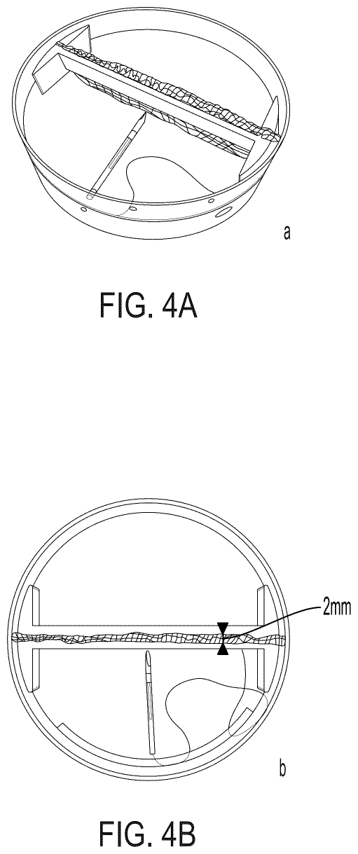

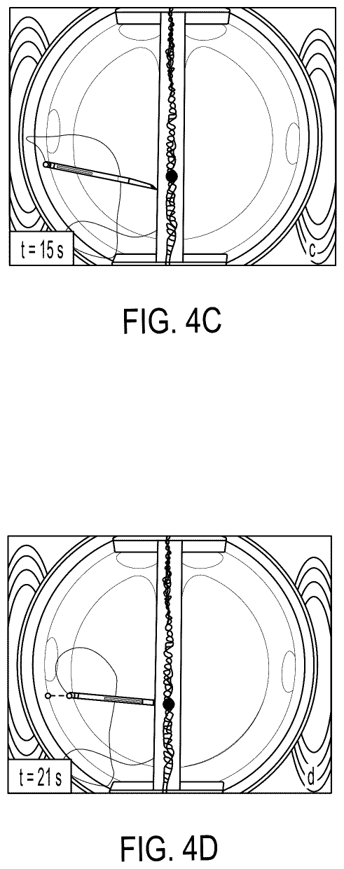

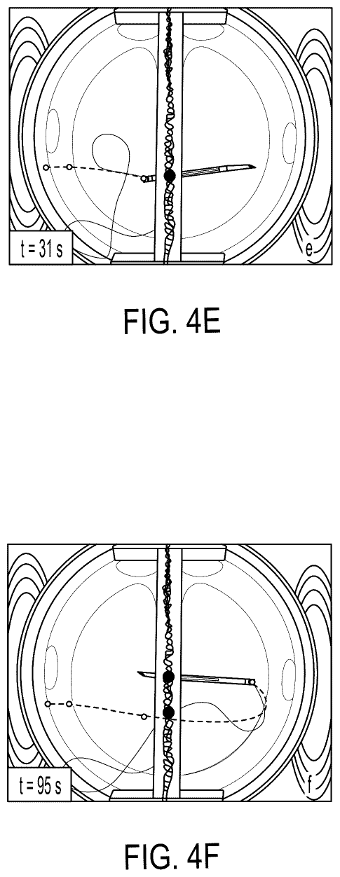

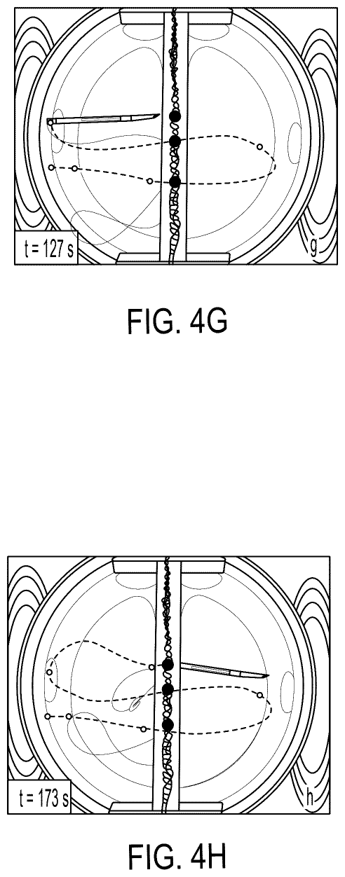

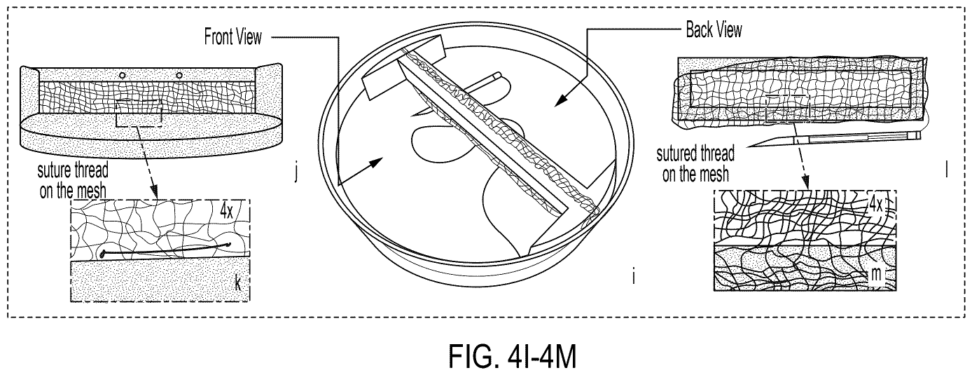

Using the MPACT-Needle and associated optimized controlling parameters T and D, we aimed to demonstrate how the significant force increase enabled the completion of challenging tissue penetration tasks. Due to the scaling laws for magnetic actuation, it is challenging to achieve sufficient force for penetration with a miniature needle using gradient pulling methods. We have experimentally characterized the required penetration forces for five different types of samples. These samples are rabbit abdominal wall, chicken breast tissue, rabbit eye, 0.6% Agar gel, and bacon strip. We have repeated each penetration experiment at least five times. Our experimental findings regarding the required penetration forces are summarized in Table 1. For the suturing and transcorneal penetrations, the MPACT-Needle provides the sufficient penetration forces. On the contrary, suturing onto rabbit abdominal wall, chicken tissue or bacon strip would require a stronger electromagnetic coil system as shown in the Table. To demonstrate the basic suturing capabilities and eye cornea access, we demonstrate (1) in vitro suturing of tissue phantoms with a mesh, and (2) ex vivo rabbit eye penetration in two different experiments. In the first experiment, we implemented a suturing task stitching a gauze mesh on an agarose gel tissue phantom, the objective being a procedure that mimics a hernia repair surgery. We prepared 0.6% agarose gel with 3 mm thickness, then covered one surface of the agarose gel with a gauze mesh. This sample was clamped vertically and placed along the centerline of the workspace. This centered clamp configuration allows us to steer the needle freely to demonstrate suturing tasks within the workspace of our magnetic system. It is important to note that the penetration forces at the start of penetration were lower since the location of the magnetic piston was at the far end of the attractive coil to penetrate. The back-and-forth hammering motion is partially automated. This partial automation takes the handheld remote controller (Xbox Controller, VOYEE) input and maps this into a hammering behavior on the magnet. By changing the polarity and current intensity of the electromagnets, we can keep the alignment of the magnet pointing towards the direction of penetration while moving the magnetic piston back and forth, with the user tuning the intensity of the backward pulling force. Both positional and orientational steering are performed manually using the handheld remote controller which controls the coils via PWM signals sent from a motor controller to the MagnetoSuture™ coils. The MPACT-Needle was able to generate more than 248 mN penetration force in average, which is found to be the penetration force required for the agarose gel used in this study. The applied running suture, consisting of 3 penetrations was completed in 158 s. Agarose gel and mesh suturing results are presented in FIG. 4.

TABLE 1
Experimentally measured penetration forces for
hypodermic needles in various types of samples.

| | Sample Type | | | | |
|---|---|---|---|---|---|
| Penetration | Rabbit | Chicken | | | |
| Force | Abdominal | Breast | Rabbit | Bacon | Agar Gel |
| Measurements | Wall | Tissue | Eye | Strip | (0.6%) |
| Needle Size (G) | 14 | 14 | 25 | 14 | 14 |
| Mean Force (N) | 0.85 | 0.55 | 0.36 | 0.46 | 0.25 |
| Std Force (N) | 0.25 | 0.23 | 0.06 | 0.11 | 0.01 |
| Experiment | 8 | 8 | 5 | 5 | 8 |
| Repetition (n) | | | | | |

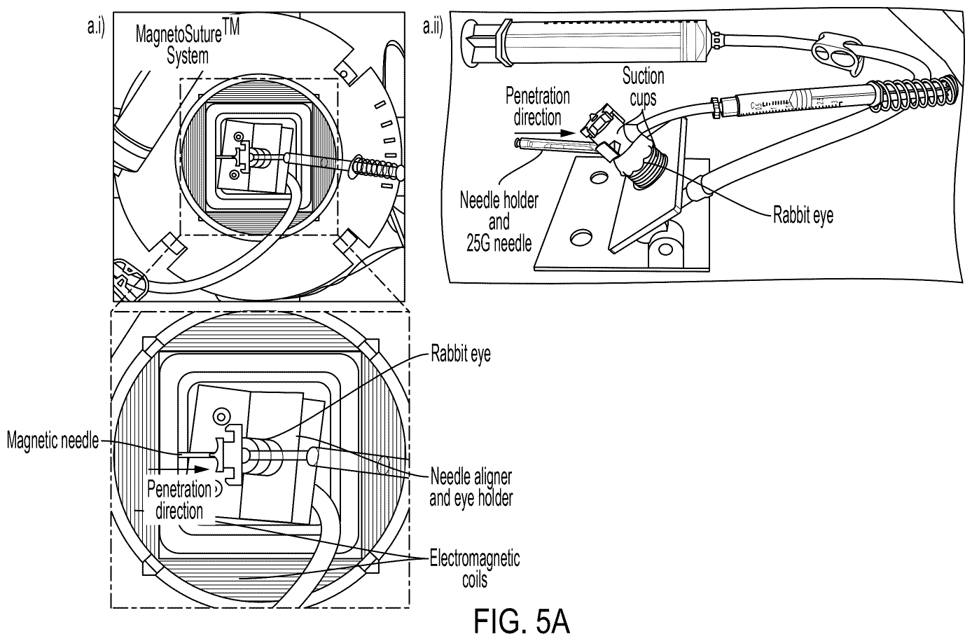

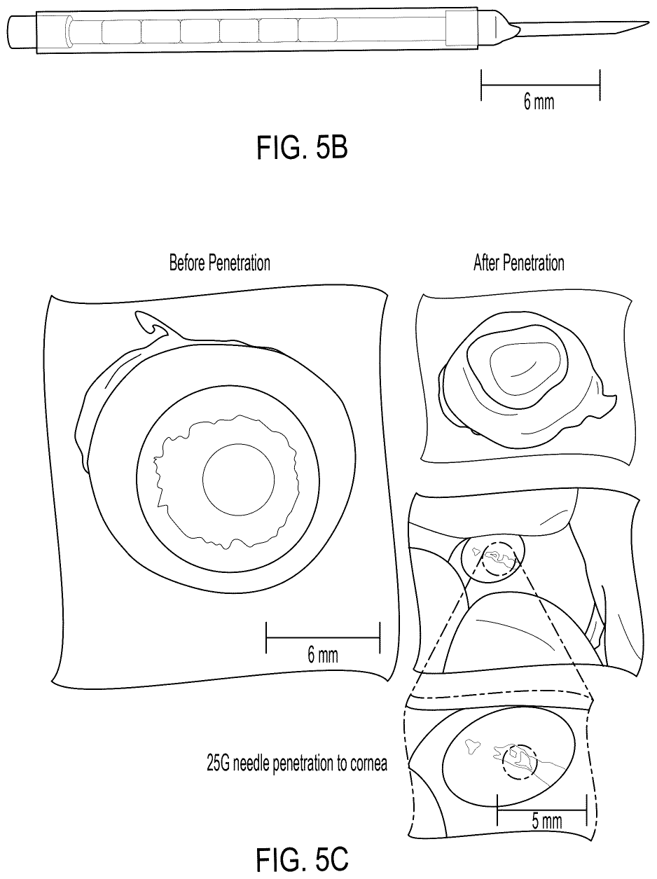

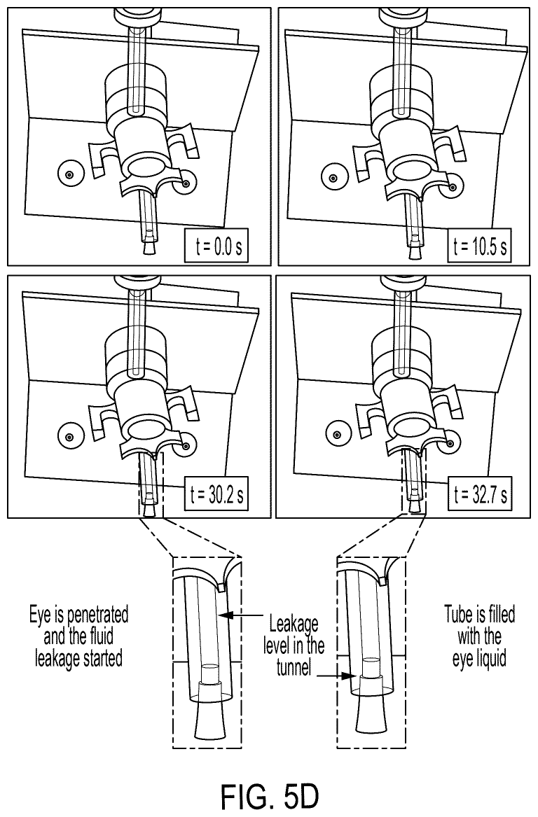

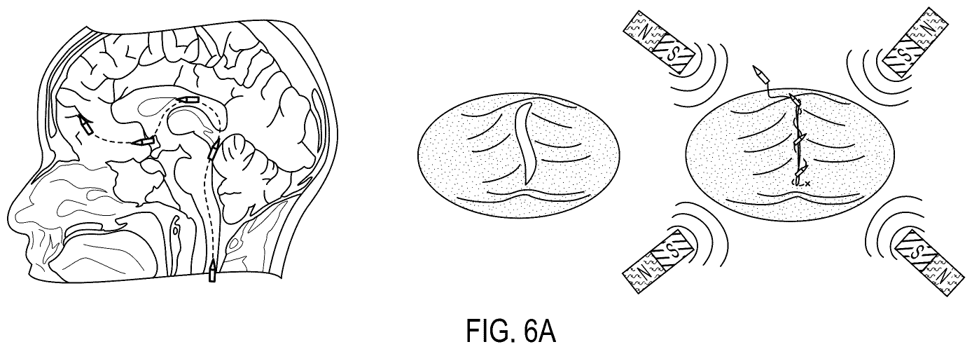

In the second experiment, a 25G MPACT-Needle was combined with a trephine to demonstrate full-thickness cornea tissue penetration using the MagnetoSuture™ System as a first step towards tissue hydrodissection and minimally invasive intraocular access in ocular surgery (FIG. 5a). A whole rabbit eye was prepared by dissecting the conjunctiva and injecting phosphate buffered saline to achieve an intraocular pressure of 20 mmHg. The eye was placed in line with the coil axes of the MagnetoSuture™ system and the trephine was secured using a clinical vacuum system (Moria Surgical, 17202D800). It is important to note that the trephine was positioned such the 25G MPACT-Needle was aligned with the coil axis to maximize the thrust of the needle (FIG. 5b). The eye was angled 30° with respect to the needle to replicate the setup used in the force characterization tests. Using a partially automated hammering strategy, the MagnetoSuture™ System achieved full thickness corneal penetration in 30.1 s (FIG. 5c), and achieved a thrust force in excess of 364.9 mN based on rabbit cornea tissue characterization tests described in the methods. After tissue penetration, the vacuum pressure was released and the trephine with MPACT-Needle was removed from the rabbit eye. Visual inspection of the cornea revealed an acute defect at the site of needle penetration, as highlighted in FIG. 5d. Once the rabbit eye was removed from the MagnetoSuture™ System, the corneal tissue collapsed indicating loss of intraocular pressure consistent with a needle puncture.

### 3 Discussion

One of the key challenges in magnetically actuated robotics is overcoming the problems of insufficient force imposed by the scaling laws of applied magnetic forces on small robotic end-effectors or devices. Miniature robots either require special designs for true penetration into a tissue, or they require very high electrical power and short distances to electromagnets that are incompatible with clinically relevant applications. While we were accomplishing up to 30 mN force using a sharpened NdFeB needle with the same electromagnetic system in our previous work, and 18 mN by only continuous magnetic gradient pulling of the MPACT-Needle, with the MPACT-Needle optimized design and actuation mechanism, we are able to accomplish up to 410 mN momentary penetration force. This mechanism and actuation technique proposed can be implemented in any other magnetic needle or magnetic tool to increase actuation forces.

In this work, we demonstrate impact-force based penetration that can significantly enhance the penetration force (22.7-fold improvement), allowing the needles to penetrate into rabbit cornea and tissue-like gels successfully. Rabbit corneas were chosen for these experiments because they exhibit similar mechanical properties to human tissue, and human cadaveric eyes were not available for testing. When accounting for needle gauge and bevel angle, the forces required to penetrate human cornea and corneoscleral limbus tissues are 282 mN and 382 mN respectively. These forces are similar to the penetration force we experimentally derived for rabbit tissues (364.9 mN), demonstrating that the MagnetoSuture™ system is capable of clinical corneal penetration and intravitreal injections. This mechanism offers an important potential route of force magnification for suturing and other needle-based interventions in clinically relevant settings, where repetitive mechanical penetration is required.

We designed a 14G hypodermic needle as the tip size of our MPACT-Needle for suturing demonstration. The MPACT-Needle for eye penetration implements a needle tip of 25G, which is a clinically standard size for eye interventions. Even though such a size is capable of demonstrating the proof-of-concept manipulation and force advantage demonstrations of the impact mechanism, further miniaturization of the needle will be required for in vivo applications. With precise manufacturing techniques, the needle size can be reduced. Combining the miniaturized needle with a more powerful electromagnetic coil system will enable penetration of stiffer tissues via the application of stronger impact forces, enabling further needle miniaturization. Additionally, more capable electromagnet arrays will enable suturing via impact-based penetration action to be performed at farther distances, making use of larger workspaces. With appropriate power electronics, larger electromagnetic coils capable of generating cyclic impact-based forces at higher frequencies (10-30 Hz range) could be designed and implemented, significantly expanding penetration possibilities.

The tissue mass moving with the needle and the rigidity of the tissue mass could change the impact force delivered via the magnetic piston and the optimized duty cycle parameters. Even though the magnetic piston does not bounce back with the free-standing MPACT-Needle design, the tissue properties in contact with the needle may result in a bouncing back behavior, resulting in differences in the impact force values and the optimized actuation sequence parameters. The mechanical responses vary from tissue to tissue, and determining the exact values would require a diligent set of simulation and characterization experiments related to the tissue properties and the needle-tissue interactions. We did not observe a critical difference in actuation sequence parameters in the demonstration experiments, but it could be more significant for stiffer and heavier tissue samples and it may require a further analysis on the tissue-needle dynamics.

We demonstrated our robot's capability on a full-thickness transcorneal penetration scenario using a 25G needle tip with a 14G needle body. While standard 25G needles are routinely used for full-thickness penetration tasks such as intravitreal injections, procedures that require partial thickness transcorneal penetration are more difficult for the surgeon to perform. Deep anterior lamellar keratoplasty for instance, requires a surgeon to place a 25G needle within 60 μm of a tissue layer for a successful transplant. Perforation of this tissue layer is catastrophic to the surgery and despite microscope visualization, occurs in up to 60% of cases for new surgeons. A magnetically controlled 25G needle tip could be an ideal solution to achieve consistent and accurate partial tissue penetration of the cornea. Therefore, incorporating precise depth control would enable the MagnetoSuture™ System to complete more challenging surgical tasks such as partial thickness corneal penetration for deep anterior lamellar keratoplasty. Depth control could be achieved by incorporating depth sensing technology within the MPACT-Needle bevel. Modeling from our previous work, it would be feasible to embed an A-scan OCT fiber and use depth feedback control, to enable micron-level positioning of the MPACT-Needle in corneal tissue using the MagnetoSuture™ system as in Opfermann et al. (In 2021 IEEE/RSJ International Conference on Intelligent Robots and Systems (IROS). IEEE, 2021 757-764) Therefore, future studies will aim to improve magnetic piston control over the impact-pulse-based actuation with more precise timing of forward and backward motions throughout the entire workspace. By using the magnetic fields, the 1-DOF transcorneal penetration experiments can be improved to a 3-DOF penetration, similar to the magnetic suturing demonstration, for more complex interventional requirements to the cornea.

The penetration demonstrations are implemented in 2D planar surfaces (3-DOF) to show the proof-of-concept without introducing technical challenges of 3D (5-DOF) manipulation such as gravitational forces and lack of surface support. The capabilities shown in this study in 2D platforms can be applicable in 3D environments as well if the necessary hardware and setup is engineered. Adding a pair of z-coils and side view cameras could be the minimum requirements to conduct 3D experiments for our system. The proposed robot design and impact mechanism would function similarly in 3D. Since many clinically relevant scenarios are in 3D, adding a z-coil pair to magnetically actuate the robot for 3D penetration is an important future step to improve this novel magnetic device and to come closer to clinical translation.

The level of autonomy proposed in this study is commanding the suturing needle via the user's handheld remote controller input. Therefore, the autonomy of the system can be represented as level 0 according to Yang et. al. (Science Robotics 2017, 2 8638) These handheld remote controller inputs can be used to rotate and translate the needles into desired poses with 3-DOF. Controlling the pulsing period for generating backward and forward cycles is implemented via user input as well. Since the magnetic field strengths are highly nonlinear with respect to distance from the field source, such subcycles of pulling and pushing should be tuned depending on the location of the needle. In other words, K_{b} and K_{f} values should be tuned individually, depending on the distance of the magnet towards the pushing and pulling coils. Such tuning can not be done manually in real-time steering. Extracting the robot position information, and using this information to compute the K_{b} and K_{f} values that provide ideal functioning of the repetitive impact-force mechanism is a must. This would advance the autonomy of the system and allow better and smoother operation of the impact-force mechanism. Improving the autonomy level would enable assistive interventional procedures, and allow full automation of routine clinical procedures such as suturing. In the experiments, we have observed difficulty in exact positional control, particularly immediately after the needle completes a penetration. In such moments, the user's response is too slow to stop and prevent the needle from hitting the walls of the petri dish. In a clinical scenario, this could introduce a safety issue. Therefore, a closed-loop control algorithm that can detect such sudden accelerations and prevent fatal movements is a must. Such autonomy can serve at an assistive level to prevent user mistakes, or to take responsibilities to conduct the tasks fully autonomously.

Assistive needle control techniques could be integrated, which could take the user input as the desired position and orientation for the needle and autonomously steer the needle into this desired position. A fully autonomous suturing needle would need to be capable of assessing and analyzing the overall suturing state. This requires advanced image processing algorithms as well as effective decision making and needle steering strategies. Lastly, proper imaging methodologies for in vivo experiments should be integrated for autonomous tasks. Such imaging methodologies can consist of laparoscopic optical cameras, OCT, ultrasound, MRI, or X-ray fluoroscopy. While future efforts will expand on these results with further ex vivo and in vivo experiments, the work presented here establishes the physical parameters for tissue penetration using our MPACT-Needle and defines needed improvements with regards to image-guidance and feedback. We envision that in vivo experiments would add additional challenges with respect to coordinate transformation, 3-D localization, and localization challenges in dynamic and cluttered surgical environments. Focusing on the challenges related to (1) the safe and precise control of the impact mechanism in 3D environments, (2) intraoperative imaging for feedback to the surgeon and for autonomous control applications, and (3) in vivo demonstrations of the MPACT-Needle are crucial next steps to translate our studies into preclinical steps.

### 4 Materials and Methods

### 4.1 Magnetic Actuation and Impact Dynamics

The magnetic pulling forces, Fₘ, and torques, Tₘ, acting on a magnetic body can be presented as

$${T}_{m} = {V}_{m} ( {M}_{m} \times B ) , \qquad ( 5 )$$

$${F}_{m} = {V}_{m} ( {M}_{m} \times ∇ ) B , \qquad ( 6 )$$

where Vₘ is the volume of the magnetic piston, Mₘ is the average magnetization of the magnetic piston, and B is the magnetic field vector generated by the electromagnetic coils. Tₘ allows rotation of the needle and pointing the needle tip in any desired direction. Fₘ provides the pulling force on the magnetic piston causing the translation and impact momentum delivery inside the tube.

A basic Coulomb friction model indicates that the surface friction of the piston on the interior surface of the tube, F_{f}, is orders of magnitude smaller than the magnetic pulling force Fₘ. Therefore, for simplicity, we neglect the surface friction in our dynamics calculations. For the magnetic piston travelling from the tail to the tip of the needle on a planar surface under a constant average pulling force, Fₘ, the Euler's law of rigid-body motion for the magnetic piston yields the equations of dynamics:

$${F}_{net} = ma = {F}_{m} + {F}_{impact} , \qquad ( 7 )$$

where Fₙₑₜ is the net force on the magnetic piston. The impact force, F_{impact} occurs only when the piston hits to the impact plate and can be computed as

$${F}_{impact} = \{ \begin{matrix}{f}_{impact} ( {V}_{rel} ) , & if ( ‖ {x}_{m} ‖ \geq {x}_{crit} ) \\ 0 , & otherwise\end{matrix} . \qquad ( 8 )$$

where xₘ is the center location of the magnetic piston, x_{crit} is the critical magnet center position where the contact to impact plate takes place, and Vᵣₑₗ is the relative velocity between the magnetic piston and the impact plate. f_{impact} can be formulated by the simplified impact physics for two rigid body collision with a constant deceleration,

$${f}_{impact} ( {V}_{rel} ) = - \frac{{mV}_{rel}}{Δ {t}_{impact}} . \qquad ( 9 )$$

As it can be seen in Equations (6), (7), and (8), there are two types of forces acting on the magnetic piston. While Fₘ is the standard magnetic pulling force, F_{impact} occurs under the presence of contact with the impact plate. The final force on the needle tip is the most important parameter for maximizing the penetration and suturing efficacy. For a needle penetrating into tissue, the environment-based torques and forces as well as magnetic forces and torques acting on the needle can be represented as

$${T}_{needle} = {T}_{m} + {T}_{d} , \qquad ( 10 )$$

$${F}_{needle} = \{ \begin{matrix}{F}_{m} - {F}_{impact} + {F}_{d} , & if ( ‖ {x}_{m} ‖ \geq {x}_{crit} ) \\ {F}_{m} + {F}_{d} , & otherwise\end{matrix} . \qquad ( 11 )$$

External forces and torques due to the interactions with the environment or tissue are represented by F_{d} and T_{d}, respectively.

### 4.2 Impact-Based Actuation Mechanism

The impact-based actuation methodology requires an actuation sequence that pulls and pushes the magnetic piston back and forth along the direction of the penetration. The momentum accumulated on the piston travelling along the tubular structure is transferred to the impact plate in a short time, causing a much stronger momentary force. This sudden impact behaviour provides a large penetration force on the magnetic needle that would not be achievable without a significant upgrade to the electromagnetic actuation hardware. This impact-based actuation mechanism is depicted in FIG. 1b.

The back and forth motion of the magnet creates a certain sequence for the magnetic field and the gradient field applied. For a magnet penetrating along a desired direction \hat{r}, the applied magnetic field and gradient as a function of time should have the following properties:

$${B}_{m} \vert \vert \hat{r} , \qquad ( 12 )$$

$${F}_{m} ( t ) = \{ \begin{matrix}{K}_{f} ‖ {F}_{max} ‖ \hat{r} , & if ( mod ( t , T ) \geq D \cdot T ) \\ - {K}_{b} ‖ {F}_{max} ‖ \hat{r} , & if ( mod ( t , T ) < D \cdot T )\end{matrix} , \qquad ( 13 )$$

where K_{f} and K_{b} are the forward and backward pulling force constants, respectively, limited between 0 and 1. Bm is the magnetic field vector generated by the coils, Fₘₐₓ is the maximum pulling force that is exerted on the magnet, t is the current time, T is the period duration for the full cycle of back-and-forth piston movement, D is the duty ratio for the forward pulling duration limited between 0 and 1. This pulling force sequence over time with relevant parameters are depicted in FIG. 2d.

In addition to the actuation parameters, the location of the needle has an important contribution. Because the magnetic field generated is not homogeneous within the workspace, i.e., the field is stronger near to the electromagnets, the backward pulling force and forward pulling force vary depending on the location of the magnet. If the needle is in line with the electromagnets and the tip is closer to the magnets than the tail, the forward pulling force is naturally stronger. In the opposite situation, where the tail of the needle is closer to the electromagnets, the backward pulling force dominates. Therefore, a handheld remote controller-based user input is continuously provided to tune D, K_{f}, and K_{b} such that the forward and backward travelling motion is satisfied throughout the entire workspace.

To characterize this impact behaviour of the needle as a function of time-related parameters D and T, we created an experimental setup with a load cell (Transducer Techniques, GSO-1K) located inside of our magnetic system (FIG. 3b). A two-dimensional sweep along D and T was implemented. We swept the range from 0.2 to 0.8 with 0.1 increments for D, and 50 ms to 250 ms with 50 ms increments for T. For each experiment, the impact motion lasted for 5 s resulting in more than 20 cycles. The force readings are used to 1) compute the average impact force generated, 2) compute the highest force density per time.

### 4.3 MagnetoSuture™ Setup

In this work, we implemented an optimized MPACT-Needle with the configuration shown in FIG. 2. To demonstrate the performance of the MPACT-Needle, we employed our physical MagnetoSuture™ system that was previously presented in and illustrated in FIG. 1c. The needle was submerged in a viscous medium made by water-glycerol mixture inside a Petri dish (diameter=85 mm). The MPACT-Needle was tele-manipulated through external magnetic fields generated by an array of four uniformly spaced cylindrical electromagnets. Each electromagnetic coil was made by approximately 12 wound layers of 54 turns of AWG 16 polyimide-coated copper wire (Nₑₘ=12×54). The inner diameter of the EM is 85 mm, their outer diameter is 98 mm (average diameter 2ρₑₘ=91.5 mm), and their length is lₑₘ=60 mm, as shown in FIG. 1c. Four identical iron cores with diameters of 52.18 mm and lengths of 66.3 mm are inserted in the electromagnetic coils for boosting the magnetic field. The electromagnets are driven by two dual channel H-bridge motor controllers (RoboClaw, Basic Micro Inc.) powered by an AC/DC converter capable of supplying 62.5 A and 48 VDC (PSE-3000-48-B, CUI Inc.). Visual feedback of the needle pose in the Petri dish was obtained by using a FLIR Blackfly camera (BFS-U3-13Y3C-C) with a resolution of 1280×1024 pixels. The workspace was illuminated by a ring light mounted on a custom 3D-printed adapter.

4.4 Teleoperation System with a Handheld Remote Controller

The handheld remote controller (Xbox Controller, VOYEE) inputs are used to control the needle motion and its penetrating impulses teleoperatively. The user commands are implemented to rotate and translate the needle on a planar surface in real-time while the operator monitors the needle via the top camera view. Moreover, the hand-held remote controller input is also being used to apply the sequence of pulling and pushing the magnetic piston along this direction based on the parameters D, T, K_{b}, and K_{f}. It is important to have a changing value for K_{b} and K_{f} since the magnetic pulling forces are spatially nonlinear. While the needle changes its position, the ideal K_{b} and K_{f} constants should be updated. This tuning of constants is accomplished by the handheld remote controller's continuous buttons during the operation of the needle. Typically, K_{b} is kept in the range that provides the oscillatory hammering behaviour while the net motion of the needle is forward.

### 4.5 Agar Gel Sample Preparation

Here, 0.6% agarose gel mimicking the stiffness of a brain tissue at 0.61 kPa elastic modulus with 3 mm thickness covered with a gauze mesh (CVS, Latex-free Gauze 5 CT) was used for the suturing experiment. A holder testbed, as shown in FIG. 4, is designed. This testbed is aimed to clamp the samples perpendicularly to the planar needle workspace. Each testbed consisted of 1) two 3D printed pieces which could form a circular horizontal platform (radius=41 mm) and a standing clamping frame (height=11 mm), 2) a Petri dish (inner radius=43 mm), and 3) glycerin-water solution (30 ml) (Glycerin Vegetable, Sanco Industries, Inc., Fort Wayne, IN). The clamping frame was positioned at the center of the Petri dish. Hence, adequate space was left for steering the needle in the suture study. Depending on the thickness of the sample, a non-sticky tape was inserted between 3D printed platform edges and Petri dish walls to close the gap and enhance the clamping functionality.

### 4.6 Rabbit Eye Experiment Preparation

Adult rabbit whole eyes were collected fresh in phosphate buffered saline (PBS) with Penicillin-Streptomycin, Amphotericin B, and Gentamicin and shipped the same day on wet ice. Prior to testing, the rabbit eye was prepared by dissecting the conjunctiva and injecting PBS until the intraocular pressure was 20 mmHg. During experiments, the rabbit eye was placed in a whole eye holder (FIG. 5a), and stabilized by applying a vacuum using a 100 ml syringe. The whole eye holder was manufactured using a 12.7 mm NPT barbed hose fitting that was epoxied to a 3D-printed stand. The holder was designed to position the apex of the eye with the central axis of the MagnetoSuture™ System's coils.

The MPACT-Needle holder was manufactured by drilling a 0.5 mm hole at a 300 angle through the lateral wall of a corneal dissection trephine. A 2 mm inner diameter polycarbonate round tube was cut to 50 mm length, aligned with the drilled hole, and epoxied to the trephine. The size of the polycarbonate tube was chosen to accommodate the diameter of the 25G MPACT-Needle, while guiding the needle bevel to the apex of the rabbit cornea. After loading the 25G MPACT-Needle, the polycarbonate tube was capped using an ABS plug. Prior to the experiments, the needle holder was manually aligned with the apex of the cornea and attached to the whole eye using a commercial trephine vacuum (Moria Surgical, 17202D800). A commercial trephine vacuum was used for all experiments so that the vacuum pressure did not exceed the clinical conditions. At the end of the experiment, the vacuum pressure was removed, releasing the trephine from the eye so that the MPACT-Needle penetration could be documented.

### 4.7 Force Characterization Measurements

A single axis load cell (Transducer Techniques, GSO-1K) is located in the workspace of the MagnetoSuture™ setup. The voltage reading from the load cell's strain gauge is transferred to a data acquisition card (National Instruments, NI-9205) at a rate of 10 kS/s. Through the interface of a LabView program, the voltage data is digitized and mapped into the force domain. Load cell characterization experiments have shown that the measurements have the sensitivity of 5.69 mN. Prior to the force measurements, the tubular structure of the needle body is attached perpendicularly to the sensing region of the load cell by using a cyanoacrylate-based adhesive.

4.8 Agar Gel with Gauze and Rabbit Eye Force Characterization

To characterize the penetration forces in the agar gel phantom tissue with gauze and rabbit eye used in our experimental study, a needle penetration force recording system was setup by using a syringe pump (PHD ULTRA™, Harvard Apparatus) as a linear motion stage. The following force characterization experiments were performed.

Agar Gel with Gauze: A 14G needle was attached to a single axis load cell (Transducer Techniques, GSO-1K), which was fixed on the moving part of the syringe pump. A 3D-printed tissue holder was placed along the needle's moving direction. For enabling repeated needle penetration tests on the same piece of tissue sample, the location of the tissue holder can be adjusted on the plane that is perpendicular to the needle's moving direction. We repeated the penetration on the same sample 5 times at various locations to generate penetration force ranges of both sample types. As a result of these experiments, we found out that the average penetration force of the agar gel with gauze is 248 mN±98 mN.

Rabbit Whole Eye: Corneal penetration forces were measured by attaching a 25G needle to the same single-axis load cell used in the agar penetration tests and mounting the system on the syringe pump. A whole rabbit eye holder was mounted at 30° with respect to the 25G needle, and the linear stage was programmed to a velocity of 0.3 mm/s. Penetration forces were recorded until the needle advanced through the corneal tissue and into the intraocular space. The maximum penetration force was defined as the peak force during testing and the average peak penetration force is found to be 364.9 mN±54.6 mN. Rabbit eyes were pressurized to 20 mmHg during all tests to minimize variance in the samples. Penetration forces for corneal tissue were measured on five samples and are reported as the average penetration force with associated standard deviation.

### 4.9 Statistical Analysis

The force characterization experiments for rabbit abdominal tissue penetration, agar gel penetration, and chicken breast tissue are repeated 8 times (n=8). Similarly, rabbit eye penetration and bacon strip penetration experiments are repeated 5 times (n=5). The mean and standard deviation calculations are computed based on the highest point of force measured. The sampling rate of the data is 1 kHz.

### Example 2: Tetherless Needle Steering in Soft Tissue Using Magnetic Impact

### 1 Introduction

Remote, untethered manipulation of surgical needles and other instruments has the potential to reduce tissue damage caused by inserted surgical instruments, further decreasing the invasiveness of such procedures. Non-contact methods of steering suture needles have been devised and used to demonstrate significant dexterity. These methods include using magnetic fields to twist magnetic corkscrew-shaped catheters, pull magnetic needles, and hammer impact-force-based magnetic needles through various tissues and tissue mimics. Of these approaches, rotational corkscrews and magnetic hammers hold the most promise, as they both enable significant, long-range penetration of dense tissues. While corkscrew-shaped devices have well-established methods for intricate steering, hammer-based needle penetration is significantly newer, and steering such hammer-based devices in matrix materials has not yet been demonstrated.

Bevel tipped steerable needles are capable of deflecting around obstacles and being steered to targets by relying on an asymmetric bevel tip that causes a deflection of the needle when inserted. These needles offer significant improvement over straight needles since they can be steered, but rely on manual or robotic insertion of the hub of the needle. Thus, they are more invasive than a tetherless needle and have the added drawbacks of requiring long needles, added stress on surrounding tissue, and the inability to turn past 90° from the initial insertion angle, making it impossible to use them for applications such as suturing.

Here we present the use of combined magnetic hammering field gradients and bevel tip deflection to demonstrate the steering of a tetherless magnetic hammer needle in a tissue phantom. This device combines the benefits of both magnetic manipulation and traditional bevel tipped needle steering. Contributions include the first demonstration of in-matrix steering for hammer-based needles, as well as the smallest hammer-based needle yet demonstrated. In this paper we will discuss the details of our design, the methods for magnetic actuation, a model for bevel-tipped steering, present experimentation characterizing the steering capabilities, and finally demonstrate the ability to drive the needle to desired target locations using human in the loop control.

### 2 Materials & Methods

### 2.1 Needle Design and Magnetic Guidance System

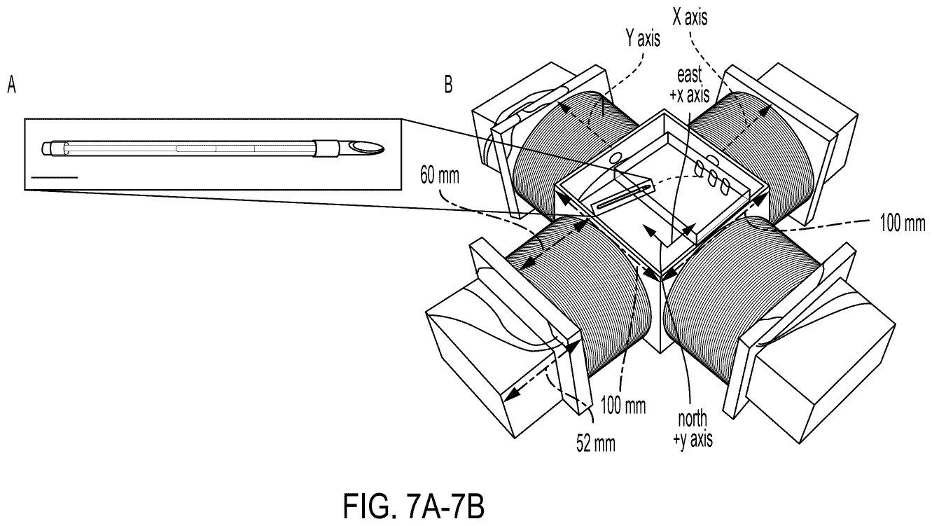

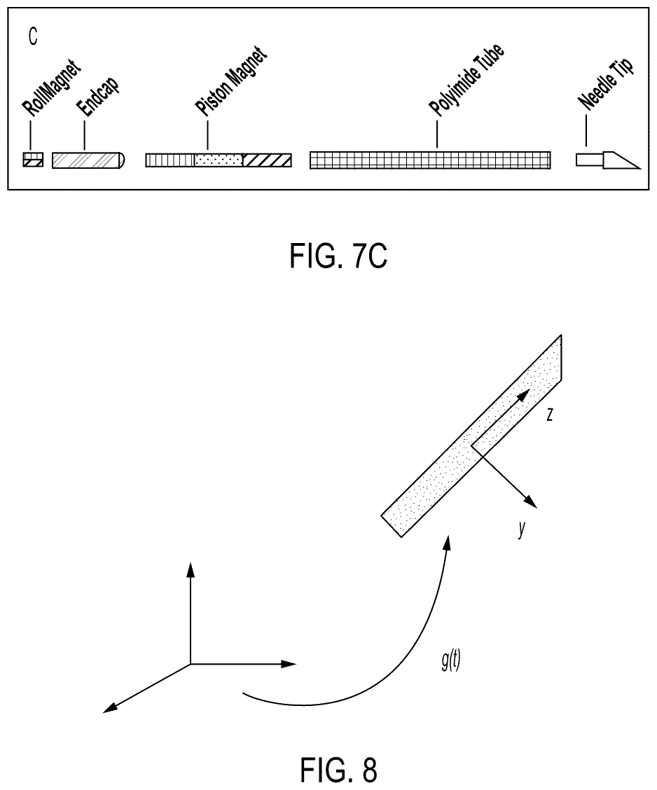

To achieve magnetic needle steering using an MPACT-needle, we construct a sharp-tipped needle with freely-sliding NdFeB magnets housed in a hollow tube. A photograph and schematic of our needle are shown in FIGS. 7A and C, respectively. The needle consists of a bevel-sharpened MRI-compatible 18G needle tip, a tubular polyimide needle shaft (1.27 mm inner diameter, 32 μm wall thickness), three NdFeB magnets placed inside the tubular needle shaft (each being 1 mm diameter, 4 mm length), and an end cap containing a diametrically magnetized permanent magnet (1 mm diameter, 1 mm length). The end cap ensures 5 mm of separation between the end magnet and internal piston magnet to avoid magnetic interaction. For the needle tip, we cut the tip from an 18G MRI-compatible biopsy needle (30° bevel angle) consisting of an inner core and outer, tri-beveled sheath. Using an MRI-compatible needle tip avoids magnetic interactions with indwelling magnets and external applied fields. The MRI-compatible needle tip (8 mm length) is inserted into the polymimide tubular segment. The polyimide tubular segment was chosen to closely match the outer diameter of the 18G needle while still maintaining ample space for NdFeB magnets to slide inside the polyimide tubular segment. The combined magnetization of indwelling magnets is 0.01 A·m². Here we choose an overall magnet length that is ⅔ the length of the tubular needle shaft chamber, as previous work determined this ratio to provide maximum impact force for our MPACT-Needle geometry. The body is then capped with a 3D printed end cap that houses a 1 mm diameter, 1 mm long, radially magnetized cylindrical NdFeB permanent magnet with a calculated dipole strength of 0.001 A·m². The overall length of the needle is approximately 35 mm.

Magnetic steering of our MPACT-needle was performed using the previously described MagnetoSuture™ System. The system consists of six orthogonally positioned electromagnets, each electromagnet being 60 mm long, with outer diameters of 98 mm and inner diameters of 85 mm. Each coil had a total of 648 turns. Motor drivers power the coils using PWM signals generated from an Arduino, with each coil being independently addressable. Experiments are performed in a custom-printed sample holder containing a sample of 0.5% agarose gel.

### 2.2 Needle Pose to Coil Currents

Electromagnetic fields are generated by six coils at a distance from the needle that moves the needle remotely. One of the findings of Maxwell's Equations is that the magnetic field generated by each coil is a continuous function of 3-D position and can be superimposed to each other. The contributions from each of the 6 electromagnetic should be orchestrated in such a way that it exerts the desired forces and torques onto the magnetic component of the needle.

For our MagnetoSuture™ System with four coils in a horizontal plane, we mathematically compute the coil currents, uC, as follows,

$${u}_{C} = [ \begin{matrix}\begin{matrix}{i}_{1} \\ {i}_{2}\end{matrix} \\ {i}_{3} \\ {i}_{4}\end{matrix} ] = {{α}_{B , F} ( P )}^{- 1} [ \begin{matrix}{B}_{des} \\ {u}_{d}\end{matrix} ] , \qquad ( 1 )$$

where in represents the coil current from each electromagnetic coil, αB,F is the actuation matrix for a given pose, P, of the needle. The pose P consists of a two-dimensional position and a one-dimensional orientation on a planar workspace, P=[xact yact fact]^{T}. Bdes is the desired magnetic field that includes both direction and intensity, Bdes=∥Bdes∥ [sin(θdes) cos(θdes)]^{T}. ud is the two-dimensional desired magnetic pulling force exerted, ud=[Fxdes Fydes]^{T}.

Computing the actuation matrix, αB,F, is a crucial process since it maps the relation between coil currents to the exerted magnetic forces and magnet alignment. The actuation matrix can be written as

$${α}_{B , F} ( P ) = [ \begin{matrix}\begin{matrix}\overline{β} ( P ) \\ {v}_{m} {Ψ}_{m}^{T} {\overline{𝔾}}_{x} ( P )\end{matrix} \\ {v}_{m} {Ψ}_{m}^{T} {\overline{𝔾}}_{y} ( P )\end{matrix} ] , \qquad ( 2 )$$

where Ψm∈R² is the magnetization vector of the magnetic needle, vm is the volume of the magnetic component, G̅x and G̅y are magnetic gradient vectors along x and y directions, i.e.,

$$[ \frac{\partial {\overline{B}}_{1} ( P )}{\partial x} \frac{\partial {\overline{B}}_{2} ( P )}{\partial x} \frac{\partial {\overline{B}}_{3} ( P )}{\partial x} \frac{\partial {\overline{B}}_{4} ( P )}{\partial x} ] and$$

$$[ \frac{\partial {\overline{B}}_{1} ( P )}{\partial y} \frac{\partial {\overline{B}}_{2} ( P )}{\partial y} \frac{\partial {\overline{B}}_{3} ( P )}{\partial y} \frac{\partial {\overline{B}}_{4} ( P )}{\partial y} ] ,$$

respectively.

The electromagnetic field contribution from every electromagnetic coil is computed for a given unit current. β̅(P) is the vector element that contains the electromagnetic field contribution from each coil for a unit current. and can be represented as

$$[ {\overline{B}}_{1} ( P ) {\overline{B}}_{2} ( P ) {\overline{B}}_{3} ( P ) {\overline{B}}_{4} ( P ) ] = \overline{β} ( P ) . \qquad ( 3 )$$

Once Eqs. (2) and (3) are computed, the coil currents uC are found by inputting the desired magnetic field, Bdes, and magnetic force, ud and merging the results found by Eqs. (2) and (3) into Eq. (1). To adjust the roll angle of the needle we merely need to use β̅ to calculate the coil currents for a desired 3D magnetic field. In this way we can create a rotating field around the roll axis of the needle. The rear “roll magnet” located in the end cap will follow this field, setting the roll angle of the needle and allow for effective steering by adjusting the position of the bevel tip as for traditional needle steering.

The electromagnetic field generated by coils has to be computed accurately to accurately compute the alpha matrix. A typical computation method uses a Dipole Model that gives a closed-form equation which eases the implementation. However, this methodology has significant errors, especially for needle poses close to the external electromagnetic coils. Therefore, to increase the accuracy significantly, we measured the magnetic field for an array of points for a unit current applied to a single coil in our MagnetoSuture System. We then used the characterization data combined with linear interpolation for intermediate locations on the workspace to pinpoint the electromagnetic contributions coming from each electromagnet that is used to compute the actuation matrix, αB,F. Further details on how to compute these values in detail are presented in our previous study. We note that there are poses within the workspace in which the a matrix becomes singular and leads to unreasonably high and rapidly changing coil currents. Efforts were made in our trials to avoid these regions and downscaling was implemented to not overload the system when this was not possible.

### 2.3 Bevel-Tip Kinematic Model

To describe the needle with an asymmetric bevel tip during suturing operations, we use a non-holonomic model, where we treat the needle as a single rigid body. In this model, we attach a body-fixed frame at the center of the needle. Let g E SE(3) denote the pose of the rigid needle. See FIG. 8 for a graphical illustration. The so-called body velocity of the needle, denoted as ξ, is written as

$$ξ = {( {g}^{- 1} \dot{g} )}^{⋁} = {( {ω}^{T} , {v}^{T} )}^{T} \qquad ( 4 )$$

where ω and v respectively denote the angular and linear part of the body velocity, and v operation extracts a vector in R6 from a generalization of a skew-symmetric matrix. Due to the asymmetric bevel tip, the motion of the needle has non-holonomic constraints. For this, we assume that the needle only moves along the local z-axis, and that it rotates about the local x-axis. Then it follows that

$$ξ = ( \begin{matrix}ω {e}_{1} \\ v {e}_{3}\end{matrix} ) \qquad ( 5 )$$

where ei (i=1,2,3) denote the standard basis vectors for R3. Here v denotes the linear speed of the needle and ω is the rate of rotation (i.e., angular speed) due to the asymmetric tip during suturing. Both parameters can be experimentally estimated, which we do in the results section.

### 2.4 Experimentation Setup

For our experiments, we used 0.5% agarose gel which has been shown to have similar properties to that of brain tissue. For our needle, we measured the insertion force to be approximately 20 mN and measured the peak hammer force to be up to 50 mN. For our trials, we start in a similar pose for each trial. This is done in an attempt to prevent discrepancies between trials, and allow the needle to follow a trajectory that does not encounter singularities or weak gradient regions. We start each trial by inserting the needle through a 3D printed jig that locates the needle approximately 20 mm left of the center with the needle halfway inserted into the agar at an angle of 85 degrees.

Bevel Tip Steering: To characterize the maximum steering capabilities of traditional bevel tipped steering with our needle, we conducted several trials and measured the pose along the length of the trial. The roll angle of the needle was kept at the same orientation throughout the length of each trial by using human-in-the-loop control. Trials were ran to a total insertion depth of roughly 3-4 cm.

Magnetic Steering: To apply magnetic steering, we left the desired force vector along the length of the needle, but altered the desired orientation of the field on the forward stroke by a few degrees in the desired direction of rotation. Experimentally we found that at an orientation offset of more than 10°, the hammer no longer operated effectively, presumably due to friction. We conducted two trials with a symmetric tipped needle to isolate the magnetic steering. For these trials, we adjusted the offset throughout the length of the trial to achieve maximum offset while still hammering effectively. We also attempted to combine magnetic and bevel tipped steering but found that it made the steering performance worse than pure bevel tip steering.

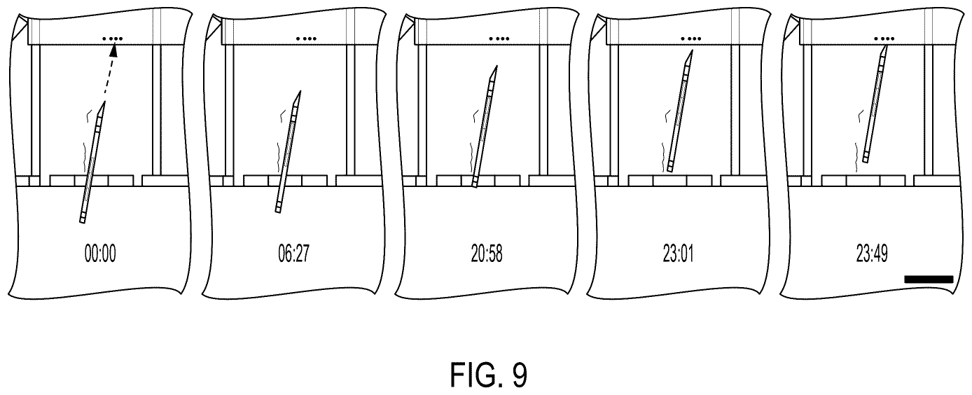

Target Trials: To validate the ability of the needle to steer to desired locations, we conducted several rounds of target trials in which we attempted to steer the needle to a target using human in the loop control. For the target trials, four targets were printed on a transparent sheet and attached to a holder at the top of the workspace so that the center of the targets was in line with the standard starting position. The targets are each 1 mm in diameter and spaced roughly 1-2 mm apart for a total spread of 5 mm from target 1 to target 4. The insertion depth was approximately 1-1.5 cm. The test set up was designed so that a fresh agar sample could be slid into the workspace between trials. See FIG. 9 for the test setup and targets. During the trials, a mix of hammer propulsion and magnetic gradient propulsion was employed by the user to propel the needle to the target. The user manually adjusted the roll angle of the needle to correct the needle's tip orientation and guide it to the targets. In total, we conducted four rounds of testing, steering to each target once per round. For each round of testing we randomized the order of targets to remove potential bias.

### 3 Results

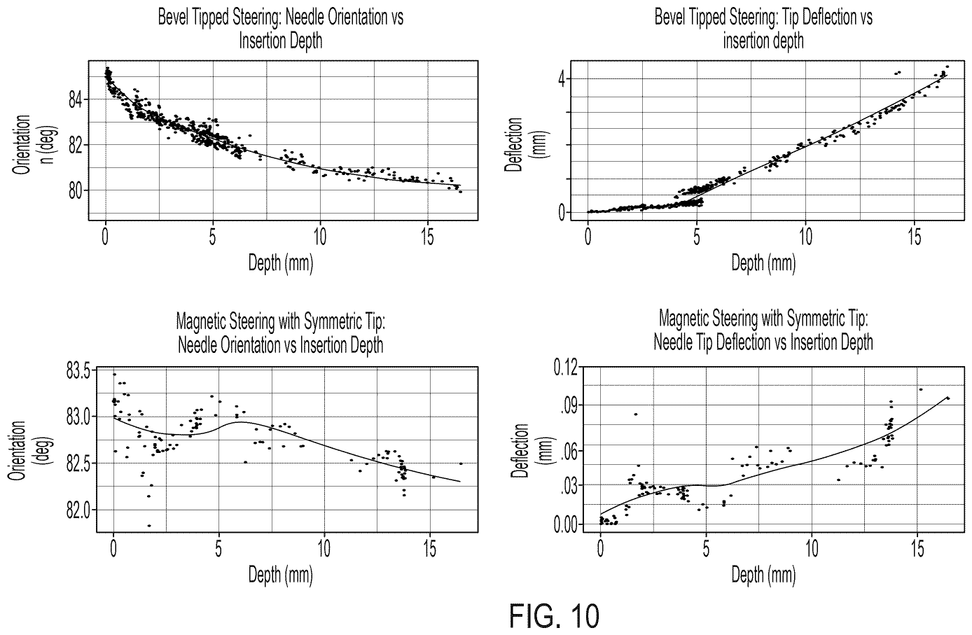

Bevel Tip Steering: FIG. 10, top, shows a representative example of the orientation change and tip deflection of a bevel tip experiment. Across all trials, the average deflection was 0.13 mm/mm (deflection/depth) for an average total deflection of 2-3 mm and the average orientation change per mm of insertion was approximately 0.21 deg/mm. This 0.21 deg/mm value corresponds to the w value in our kinematic model. The average insertion speed v was found to be 0.02 mm/s. In the figure, the decreasing orientation value corresponds to a clockwise rotation within the system.

Magnetic Steering: FIG. 10, bottom, shows the results from a trial in which we only used magnetic torque to try to steer a needle with a symmetric tip. Average rotation across all trials was 0.06 deg/mm and average tip deflection was 0.04 mm/mm.

Target Trials: Table 1 shows the error results for the target trials. A negative value represents a miss to the left of the target and a positive a miss to the right. Errors were calculated as the distance between the needle tip and the center of the desired target.

TABLE 1
Target Trial Errors (mm)

| Trial | 1 | 2 | 3 | 4 | 5 | 6 | 7 | 8 | 9 | 10 | 11 | 12 | 13 | 14 | 15 | Mean | StdDev |
|---|---|---|---|---|---|---|---|---|---|---|---|---|---|---|---|---|---|
| Signed Error | −2.7 | −3.1 | 2.8 | 1.8 | −2.8 | −3.4 | −2.6 | 3.6 | 1.3 | 2.1 | 0.5 | 0.7 | 2.7 | 2.2 | −2.5 | 0.1 | 2.6 |
| Absolute Error | 2.7 | 3.1 | 2.8 | 1.8 | 2.8 | 3.4 | 2.6 | 3.6 | 1.3 | 2.1 | 0.5 | 0.7 | 2.7 | 2.2 | 2.5 | 2.3 | 0.9 |

### 4 Discussion

Based on the results of steering characterization trials, bevel tipped steering proved to be the most effective form of steering through the agar gel. Our trials with the symmetric needle tip showed that magnetic steering is not effective on its own, and, further, when combined with bevel tip steering, actually decreases steering performance. We believe this is due to only applying a torque on the internal magnet and not along the length of the needle. The results from the bevel tipped steering and target trials, however, are promising. We show through the bevel tip trials that we can get a consistent steering capability of 0.21 deg/mm. We were then able to use this to effectively steer to 4 targets, repeatedly, with an average absolute error of 2.3 mm.

Our design does have some limitations. The main limitation is the limited steering capabilities. In order to perform sutures in the body, our design will need to be improved to make tighter turns. This can be achieved by shortening the length of the needle and utilizing a more flexible needle tube. However, this will require a system that is capable of providing larger magnetic gradients to offset the loss in volume and associated dipole strength of the needle's internal magnet. A larger, more sophisticated MagnetoSuture system would also reduce the limitations of singularities and allow for use of more of the workspace with faster insertion times. Another major limitation is the human-in the-loop aspect of the system, which contributed to the high error in the targeting trials.

### 5 Conclusion

In this example we present a novel tetherless steerable magnetic pulse actuated tapping (MPACT) needle for suturing applications in soft tissue. We demonstrate our device in 0.5% agar gel through bevel tip steering trials where we achieved and average rotation of 0.27 deg and 0.07 mm of tip deflection per 1 mm of penetration. We experimented with magnetic and magnetic assisted steering, but ultimately found it to be ineffective. Using bevel tipped steering and MPACT propulsion, we were able to steer to multiple targets using human in-the-loop control with an average accuracy of 2.3 mm. These results demonstrate the concept and are a significant step towards ultra minimally invasive suturing.

Some further aspects are defined in the following clauses:

Clause 1: An impact-force medical device, comprising: a body structure that comprises a channel disposed at least partially within the body structure, which channel communicates with first and second ends of the body structure; a medical end-effector element directly or indirectly operably connected, or connectable, to at least the first end of the body structure; and, an asymmetric magnetic element at least partially disposed within the channel, which asymmetric magnetic element is configured to selectively move within the channel at least when a magnetic field, pulse, and/or gradient is applied to the asymmetric magnetic element such that the asymmetric magnetic element reversibly applies an impulse force to the first or the second end of the body structure, which impulse force effects movement of the medical device.

Clause 2: The impact-force medical device of Clause 1, wherein the body structure is substantially rigid.

Clause 3: The impact-force medical device of Clause 1 or Clause 2, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, and wherein the impact-force medical device further comprises a steering magnet structure connected at least proximal to the second end of the body structure, which steering magnet structure comprises a dipole that is oriented substantially perpendicular to the needle tip axis.

Clause 4: The impact-force medical device of any one of the preceding Clauses 1-3, wherein the channel comprises a cross-sectional shape selected from the group of: a circle, an oval, a regular n-sided polygon, and an irregular n-sided polygon.

Clause 5: The impact-force medical device of any one of the preceding Clauses 1-4, wherein the channel comprises a substantially cylindrical form.

Clause 6: The impact-force medical device of any one of the preceding Clauses 1-5, wherein the channel comprises dimensions that maximize a momentum of the asymmetric magnetic element at points of impact of the asymmetric magnetic element with the first and second ends of the body structure when the asymmetric magnetic element selectively moves within the channel.

Clause 7: The impact-force medical device of any one of the preceding Clauses 1-6, wherein a ratio between a length of the asymmetric magnetic element and a length of the channel comprises between about 0.6 and about 0.7.

Clause 8: The impact-force medical device of any one of the preceding Clauses 1-7, wherein a length of the asymmetric magnetic element is about 12.1 mm and a length of the channel is about 18.1 mm.

Clause 9: The impact-force medical device of any one of the preceding Clauses 1-8, wherein the body structure comprises a substantially tubular form.

Clause 10: The impact-force medical device of any one of the preceding Clauses 1-9, wherein the medical device is configured to wirelessly operate as a component of a magnetic robotics system.

Clause 11: The impact-force medical device of any one of the preceding Clauses 1-10, wherein the medical end-effector element is configured to contact and/or penetrate in vitro, in vivo, and/or ex vivo tissue of a subject.

Clause 12: The impact-force medical device of any one of the preceding Clauses 1-11, wherein the impulse force effects movement of the medical device in directions that are substantially parallel to a movement pathway of the asymmetric magnetic element within the channel at least proximal to the first or second ends of the body structure.

Clause 13: The impact-force medical device of any one of the preceding Clauses 1-12, wherein the asymmetric magnetic element comprises a permanent magnet.

Clause 14: The impact-force medical device of any one of the preceding Clauses 1-13, wherein the asymmetric magnetic element comprises a magnetizable material.

Clause 15: The impact-force medical device of any one of the preceding Clauses 1-14, wherein the asymmetric magnetic element comprises a cross-sectional shape selected from the group of: a circle, an oval, a regular n-sided polygon, and an irregular n-sided polygon.

Clause 16: The impact-force medical device of any one of the preceding Clauses 1-15, wherein the asymmetric magnetic element comprises a substantially cylindrical form.

Clause 17: The impact-force medical device of any one of the preceding Clauses 1-16, wherein the asymmetric magnetic element comprises a diameter of about 1.5 mm and a length of about 12.1 mm.

Clause 18: The impact-force medical device of any one of the preceding Clauses 1-17, wherein the asymmetric magnetic element comprises neodymium iron boron (NdFeB).

Clause 19: The impact-force medical device of any one of the preceding Clauses 1-18, wherein the asymmetric magnetic element is structured such that the medical device comprises two, three, four, five, or six degrees of freedom (DOF) when one or more magnetic fields, pulses, and/or gradients are applied to the asymmetric magnetic element.

Clause 20: The impact-force medical device of any one of the preceding Clauses 1-19, further comprising an impact transmission structure operably connected to the body structure at least proximal to the first end of the body structure, wherein the impact transmission structure is operably connected, or connectable, to the medical end-effector element such that the medical end-effector element is at least indirectly operably connected, or connectable, to the first end of the body structure via the impact transmission structure and wherein the impact transmission structure is configured to transmit the impulse force to the medical end-effector element when the asymmetric magnetic element reversibly applies the impulse force to the first end of the body structure.

Clause 21: The impact-force medical device of any one of the preceding Clauses 1-20, wherein the first end of the body structure comprises a first opening that communicates with the channel, which first opening communicates with the impact transmission structure such that the asymmetric magnetic element contacts the impact transmission structure when the asymmetric magnetic element reversibly applies the impulse force to the first end of the body structure.

Clause 22: The impact-force medical device of any one of the preceding Clauses 1-21, wherein the impact transmission structure comprises an impact transmission plate.

Clause 23: The impact-force medical device of any one of the preceding Clauses 1-22, wherein the medical end-effector element comprises a needle, a suture thread, a syringe, a blade, or a combination thereof.

Clause 24: The impact-force medical device of any one of the preceding Clauses 1-23, wherein the needle comprises a hypodermic needle that comprises a door structure that selectively moves between an open position and a closed position relative to a hollow opening of the hypodermic needle.

Clause 25: The impact-force medical device of any one of the preceding Clauses 1-24, wherein the body structure further comprises a second opening that communicates with the channel.

Clause 26: The impact-force medical device of any one of the preceding Clauses 1-25, further comprising a retaining structure operably connected, or connectable, to the body structure, which retaining structure is configured to prevent the asymmetric magnetic element from passing through the second opening of the body structure and to receive the impulse force from the asymmetric magnetic element to effect movement of the medical device when the asymmetric magnetic element reversibly applies the impulse force to the second end of the body structure.

Clause 27: The impact-force medical device of any one of the preceding Clauses 1-26, wherein the retaining structure comprises a cap that at least partially closes the second opening of the body structure.

Clause 28: A kit comprising the medical device of any one of the preceding Clauses 1-27.

Clause 29: A method of penetrating a tissue of a subject, the method comprising: positioning an impact-force medical device in a first position relative to the tissue of the subject using at least a first applied magnetic field, pulse, and/or gradient, wherein the impact-force medical device comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel; and, contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

Clause 30: The method of Clause 29, wherein the body structure is substantially rigid.

Clause 31: The method of Clause 29 or Clause 30, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the method further comprises steering the medical end-effector element using at least the steering magnet structure.

Clause 32: The method of any one of the preceding Clauses 29-31, wherein the first applied magnetic field, pulse, and/or gradient and the second applied magnetic field, pulse, and/or gradient are different from one another.

Clause 33: The method of any one of the preceding Clauses 29-32, wherein the positioning step comprises moving the impact-force medical device using one or more translational motions and/or one or more rotational motions.

Clause 34: The method of any one of the preceding Clauses 29-33, wherein the positioning step and/or the contacting step comprises moving the asymmetric magnetic element into contact with the first end and/or the second end of the body structure one or more times.

Clause 35: The method of any one of the preceding Clauses 29-34, wherein the contacting step comprises moving the impact-force medical device entirely through the tissue of the subject.

Clause 36: The method of any one of the preceding Clauses 29-35, further comprising positioning the impact-force medical device in a second position relative to the tissue of the subject using at least a third applied magnetic field, pulse, and/or gradient.

Clause 37: The method of any one of the preceding Clauses 29-36, wherein the first applied magnetic field, pulse, and/or gradient and the second applied magnetic field, pulse, and/or gradient are different from one another.

Clause 38: The method of any one of the preceding Clauses 29-37, wherein the medical end-effector element comprises a needle and a suture thread, and wherein the method comprises suturing the tissue of the subject using the needle and the suturing thread.

Clause 39: The method of any one of the preceding Clauses 29-38, wherein the medical end-effector element comprises a needle and a syringe, and wherein the method comprises delivering a therapeutic agent to the tissue of the subject using the needle and the syringe.

Clause 40: The method of any one of the preceding Clauses 29-39, wherein the medical end-effector element comprises a blade, and wherein the method comprises making an incision in the tissue of the subject using the blade.

Clause 41: The method of any one of the preceding Clauses 29-40, wherein the medical end-effector element comprises a needle, and wherein the method comprises obtaining a cell or tissue sample from the tissue of the subject using the needle.

Clause 42: The method of any one of the preceding Clauses 29-41, wherein a user of the impact-force medical device is at a location that is remote from the impact-force medical device.

Clause 43: A system, comprising: an impact-force medical device that comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel; a magnetic coil subassembly configured to apply magnetic fields, pulses, and/or gradients at least to the asymmetric magnetic element; a controller that comprises, or is capable of accessing, computer readable media comprising non-transitory computer-executable instructions which, when executed by at least one electronic processor, perform at least: positioning the impact-force medical device in a first position relative to a tissue of a subject using at least a first magnetic field, pulse, and/or gradient applied by the magnetic coil subassembly; and contacting the asymmetric magnetic element with at least the first end of the body structure one or more times with sufficient impact-force using at least a second magnetic field, pulse, and/or gradient applied by the magnetic coil subassembly such that at least the medical end-effector element at least partially penetrates the tissue of the subject.

Clause 44: The system of Clause 43, wherein the body structure is substantially rigid.

Clause 45: The system of Clause 43 or Clause 44, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the non-transitory computer-executable instructions which, when executed by the electronic processor, further perform at least: steering the medical end-effector element using at least the steering magnet structure.

Clause 46: The system of any one of the preceding Clauses 43-45, wherein the non-transitory computer-executable instructions which, when executed by the electronic processor, further perform at least: actuating the magnetic field to generate a torque on the impact-force medical device.

Clause 47: The system of any one of the preceding Clauses 43-46, wherein the non-transitory computer-executable instructions which, when executed by the electronic processor, further perform at least: applying the magnetic field and/or pulse such that the impact-force medical device moves toward the magnetic coil subassembly.

Clause 48: The system of any one of the preceding Clauses 43-47, wherein the non-transitory computer-executable instructions which, when executed by the electronic processor, further perform at least: accelerating the asymmetric magnetic element of the impact-force medical device toward the first or the second end of the body structure depending on a direction of the applied magnetic field using the magnetic coil subassembly.

Clause 49: The system of any one of the preceding Clauses 43-48, wherein the non-transitory computer-executable instructions which, when executed by the electronic processor, further perform at least: adjusting a pulsing duty ratio of the applied magnetic field and a pulsing frequency of the applied magnetic field to maximize a momentum of the asymmetric magnetic element at points of impact of the asymmetric magnetic element with the first and second ends of the body structure when the asymmetric magnetic element selectively moves within the channel using the magnetic coil subassembly.

Clause 50: The system of any one of the preceding Clauses 43-49, wherein the non-transitory computer-executable instructions which, when executed by the electronic processor, further perform at least: applying a magnetic field pulsing sequence with a frequency of about 6 Hz and having a duty ratio of about 0.5 using the magnetic coil subassembly.

Clause 51: The system of any one of the preceding Clauses 43-50, wherein the non-transitory computer-executable instructions which, when executed by the electronic processor, further perform at least: applying a magnetic field strength using the magnetic coil subassembly that accelerates the asymmetric magnetic element at points of impact of the asymmetric magnetic element with the first and second ends of the body structure having an impact force at least 20 times greater than when the medical end-effector element is only pulled using a same magnetic field strength.

Clause 52: The system of any one of the preceding Clauses 43-51, wherein the non-transitory computer-executable instructions which, when executed by the electronic processor, further perform at least: generating a penetration force of the asymmetric magnetic element at points of impact of the asymmetric magnetic element with the first and second ends of the body structure of about 410 mN using the magnetic coil subassembly.

Clause 53: The system of any one of the preceding Clauses 43-52, wherein the channel comprises a cross-sectional shape selected from the group of: a circle, an oval, a regular n-sided polygon, and an irregular n-sided polygon.

Clause 54: The system of any one of the preceding Clauses 43-53, wherein the channel comprises a substantially cylindrical form.

Clause 55: The system of any one of the preceding Clauses 43-54, wherein the channel comprises dimensions that maximize a momentum of the asymmetric magnetic element at points of impact of the asymmetric magnetic element with the first and second ends of the body structure when the asymmetric magnetic element selectively moves within the channel.

Clause 56: The system of any one of the preceding Clauses 43-55, wherein a ratio between a length of the asymmetric magnetic element and a length of the channel comprises between about 0.6 and about 0.7.

Clause 57: The system of any one of the preceding Clauses 43-56, wherein a length of the asymmetric magnetic element is about 12.1 mm and a length of the channel is about 18.1 mm.

Clause 58: The system of any one of the preceding Clauses 43-57, wherein the body structure comprises a substantially tubular form.

Clause 59: The system of any one of the preceding Clauses 43-58, wherein the medical end-effector element is configured to contact and/or penetrate in vitro, in vivo, and/or ex vivo tissue of a subject.

Clause 60: The system of any one of the preceding Clauses 43-59, wherein the impulse force effects movement of the medical device in directions that are substantially parallel to a movement pathway of the asymmetric magnetic element within the channel at least proximal to the first or second ends of the body structure.

Clause 61: The system of any one of the preceding Clauses 43-60, wherein the asymmetric magnetic element comprises a permanent magnet.

Clause 62: The system of any one of the preceding Clauses 43-61, wherein the asymmetric magnetic element comprises a magnetizable material.

Clause 63: The system of any one of the preceding Clauses 43-62, wherein the asymmetric magnetic element comprises a cross-sectional shape selected from the group of: a circle, an oval, a regular n-sided polygon, and an irregular n-sided polygon.

Clause 64: The system of any one of the preceding Clauses 43-63, wherein the asymmetric magnetic element comprises a substantially cylindrical form.

Clause 65: The system of any one of the preceding Clauses 43-64, wherein the asymmetric magnetic element comprises a diameter of about 1.5 mm and a length of about 12.1 mm.

Clause 66: The system of any one of the preceding Clauses 43-65, wherein the asymmetric magnetic element comprises neodymium iron boron (NdFeB).

Clause 67: The system of any one of the preceding Clauses 43-66, wherein the asymmetric magnetic element is structured such that the medical device comprises two, three, four, five, or six degrees of freedom (DOF) when one or more magnetic fields, pulses, and/or gradients are applied to the asymmetric magnetic element.

Clause 68: The system of any one of the preceding Clauses 43-67, further comprising an impact transmission structure operably connected to the body structure at least proximal to the first end of the body structure, wherein the impact transmission structure is operably connected, or connectable, to the medical end-effector element such that the medical end-effector element is at least indirectly operably connected, or connectable, to the first end of the body structure via the impact transmission structure and wherein the impact transmission structure is configured to transmit the impulse force to the medical end-effector element when the asymmetric magnetic element reversibly applies the impulse force to the first end of the body structure.

Clause 69: The system of any one of the preceding Clauses 43-68, wherein the first end of the body structure comprises a first opening that communicates with the channel, which first opening communicates with the impact transmission structure such that the asymmetric magnetic element contacts the impact transmission structure when the asymmetric magnetic element reversibly applies the impulse force to the first end of the body structure.

Clause 70: The system of any one of the preceding Clauses 43-69, wherein the impact transmission structure comprises an impact transmission plate.

Clause 71: The system of any one of the preceding Clauses 43-70, wherein the medical end-effector element comprises a needle, a suture thread, a syringe, a blade, or a combination thereof.

Clause 72: The system of any one of the preceding Clauses 43-71, wherein the needle comprises a hypodermic needle that comprises a door structure that selectively moves between an open position and a closed position relative to a hollow opening of the hypodermic needle.

Clause 73: The system of any one of the preceding Clauses 43-72, wherein the body structure further comprises a second opening that communicates with the channel.

Clause 74: The system of any one of the preceding Clauses 43-73, further comprising a retaining structure operably connected, or connectable, to the body structure, which retaining structure is configured to prevent the asymmetric magnetic element from passing through the second opening of the body structure and to receive the impulse force from the asymmetric magnetic element to effect movement of the medical device when the asymmetric magnetic element reversibly applies the impulse force to the second end of the body structure.

Clause 75: The system of any one of the preceding Clauses 43-74, wherein the retaining structure comprises a cap that at least partially closes the second opening of the body structure.

Clause 76: A computer readable media comprising non-transitory computer executable instruction which, when executed by at least electronic processor, perform at least: positioning an impact-force medical device in a first position relative to a tissue of a subject using at least a first applied magnetic field, pulse, and/or gradient, wherein the impact-force medical device comprises: a body structure that comprises a channel disposed at least partially within the body structure, which channel communicates with first and second ends of the body structure; a medical end-effector element directly or indirectly operably connected to at least the first end of the body structure; and an asymmetric magnetic element movably disposed within the channel; and, contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. An impact-force medical device, comprising:
a body structure that comprises a channel disposed at least partially within the body structure, which channel communicates with first and second ends of the body structure;
a medical end-effector element directly or indirectly operably connected, or connectable, to at least the first end of the body structure; and,
an asymmetric magnetic element at least partially disposed within the channel, which asymmetric magnetic element is configured to selectively move within the channel at least when a magnetic field, pulse, and/or gradient is applied to the asymmetric magnetic element such that the asymmetric magnetic element reversibly applies an impulse force to the first or the second end of the body structure, which impulse force effects movement of the medical device.

2. (canceled)

3. The impact-force medical device of claim 1, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, and wherein the impact-force medical device further comprises a steering magnet structure connected at least proximal to the second end of the body structure, which steering magnet structure comprises a dipole that is oriented substantially perpendicular to the needle tip axis.

4. (canceled)

5. (canceled)

6. The impact-force medical device of claim 1, wherein the channel comprises dimensions that maximize a momentum of the asymmetric magnetic element at points of impact of the asymmetric magnetic element with the first and second ends of the body structure when the asymmetric magnetic element selectively moves within the channel.

7. The impact-force medical device of claim 1, wherein a ratio between a length of the asymmetric magnetic element and a length of the channel comprises between about 0.6 and about 0.7.

8. The impact-force medical device of claim 1, wherein a length of the asymmetric magnetic element is about 12.1 mm and a length of the channel is about 18.1 mm.

9. (canceled)

10. (canceled)

11. The impact-force medical device of claim 1, wherein the medical end-effector element is configured to contact and/or penetrate in vitro, in vivo, and/or ex vivo tissue of a subject.

12.-19. (canceled)

20. The impact-force medical device of claim 1, further comprising an impact transmission structure operably connected to the body structure at least proximal to the first end of the body structure, wherein the impact transmission structure is operably connected, or connectable, to the medical end-effector element such that the medical end-effector element is at least indirectly operably connected, or connectable, to the first end of the body structure via the impact transmission structure and wherein the impact transmission structure is configured to transmit the impulse force to the medical end-effector element when the asymmetric magnetic element reversibly applies the impulse force to the first end of the body structure.

21.-28. (canceled)

29. A method of penetrating a tissue of a subject, the method comprising:
positioning an impact-force medical device in a first position relative to the tissue of the subject using at least a first applied magnetic field, pulse, and/or gradient, wherein the impact-force medical device comprises:
a body structure that comprises a channel disposed at least partially within the body structure, which channel communicates with first and second ends of the body structure;
a medical end-effector element directly or indirectly operably connected to at least the first end of the body structure; and
an asymmetric magnetic element movably disposed within the channel; and,
contacting the asymmetric magnetic element with at least the first end of the body structure one or more times with sufficient impact-force using at least a second applied magnetic field, pulse, and/or gradient such that at least the medical end-effector element at least partially penetrates the tissue of the subject.

30. (canceled)

31. The method of claim 29, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the method further comprises steering the medical end-effector element using at least the steering magnet structure.

32. (canceled)

33. (canceled)

34. The method of claim 29, wherein the positioning step and/or the contacting step comprises moving the asymmetric magnetic element into contact with the first end and/or the second end of the body structure one or more times.

35. (canceled)

36. The method of claim 29, further comprising positioning the impact-force medical device in a second position relative to the tissue of the subject using at least a third applied magnetic field, pulse, and/or gradient.

37. (canceled)

38. The method of claim 29, wherein the medical end-effector element comprises a needle and a suture thread, and wherein the method comprises suturing the tissue of the subject using the needle and the suturing thread.

39. The method of claim 29, wherein the medical end-effector element comprises a needle and a syringe, and wherein the method comprises delivering a therapeutic agent to the tissue of the subject using the needle and the syringe.

40. The method of claim 29, wherein the medical end-effector element comprises a blade, and wherein the method comprises making an incision in the tissue of the subject using the blade.

41. The method of claim 29, wherein the medical end-effector element comprises a needle, and wherein the method comprises obtaining a cell or tissue sample from the tissue of the subject using the needle.

42. (canceled)

43. A system, comprising:
an impact-force medical device that comprises:
a body structure that comprises a channel disposed at least partially within the body structure, which channel communicates with first and second ends of the body structure;
a medical end-effector element directly or indirectly operably connected to at least the first end of the body structure; and
an asymmetric magnetic element movably disposed within the channel;
a magnetic coil subassembly configured to apply magnetic fields, pulses, and/or gradients at least to the asymmetric magnetic element;
a controller that comprises, or is capable of accessing, computer readable media comprising non-transitory computer-executable instructions which, when executed by at least one electronic processor, perform at least:
positioning the impact-force medical device in a first position relative to a tissue of a subject using at least a first magnetic field, pulse, and/or gradient applied by the magnetic coil subassembly; and
contacting the asymmetric magnetic element with at least the first end of the body structure one or more times with sufficient impact-force using at least a second magnetic field, pulse, and/or gradient applied by the magnetic coil subassembly such that at least the medical end-effector element at least partially penetrates the tissue of the subject.

44. (canceled)

45. The system of claim 43, wherein the medical end-effector element comprises an asymmetric non-magnetic needle tip having a needle tip axis, wherein the body structure is at least partially flexible, wherein the asymmetric magnetic element comprises a magnetic piston comprising a dipole that is oriented substantially parallel to the needle tip axis, wherein the impact-force medical device further comprises a steering magnet structure connected at least proximal to the second end of the body structure, which steering magnet structure comprises a dipole that is oriented substantially perpendicular to the needle tip axis, and wherein the non-transitory computer-executable instructions which, when executed by the electronic processor, further perform at least: steering the medical end-effector element using at least the steering magnet structure.

46.-48. (canceled)

49. The system of claim 43, wherein the non-transitory computer-executable instructions which, when executed by the electronic processor, further perform at least: adjusting a pulsing duty ratio of the applied magnetic field and a pulsing frequency of the applied magnetic field to maximize a momentum of the asymmetric magnetic element at points of impact of the asymmetric magnetic element with the first and second ends of the body structure when the asymmetric magnetic element selectively moves within the channel using the magnetic coil subassembly.

50. (canceled)

51. The system of claim 43, wherein the non-transitory computer-executable instructions which, when executed by the electronic processor, further perform at least: applying a magnetic field strength using the magnetic coil subassembly that accelerates the asymmetric magnetic element at points of impact of the asymmetric magnetic element with the first and second ends of the body structure having an impact force at least 20 times greater than when the medical end-effector element is only pulled using a same magnetic field strength.

52. The system of claim 43, wherein the non-transitory computer-executable instructions which, when executed by the electronic processor, further perform at least: generating a penetration force of the asymmetric magnetic element at points of impact of the asymmetric magnetic element with the first and second ends of the body structure of about 410 mN using the magnetic coil subassembly.

53.-76. (canceled)
